(19) United States
(12) Patent Application Publication
(10) Pub. No.: US 2016/0186156 A1
(43) Pub. Date: Jun. 30, 2016

(54) ARTIFICIAL CELLULOSOMES COMPRISING MULTIPLE SCAFFOLDS AND USES THEREOF IN BIOMASS DEGRADATION

(71) Applicant: YEDA RESEARCH AND DEVELOPMENT CO., LTD., Rehovot (IL)

(72) Inventors: Edward A. BAYER, Ramot-HaShavim (IL); Yael VAZANA, Rehovot (IL); Yoav BARAK, Rehovot (IL); Johanna STERN, Rehovot (IL); Hadar GILARY, Rehovot (IL); Sarah MORAIS, Rehovot (IL)

(21) Appl. No.: 14/910,001

(22) PCT Filed: Aug. 3, 2014
(86) PCT No.: PCT/IL2014/050700

§ 371 Date: Feb. 4, 2016

Related U.S. Application Data
(60) Provisional application No. 61/862,019, filed on Aug. 4, 2013.

(51) Int. Cl. C12N 9/42; C12P 19/02; C12N 9/96; C12P 19/14

(52) U.S. Cl. CPC C12N 9/2437; C12P 19/14; C12P 19/02; C12N 9/96; C12Y 302/01004

(57) ABSTRACT

Multi-enzyme complexes comprising an array of scaffold subunits designed for efficient integration of a plurality of carbohydrate-active enzymes are provided.

### FIELD OF THE INVENTION

The present invention relates to artificial cellulosome complexes comprising an array of scaffold subunits designed for efficient integration of a plurality of carbohydrate-active enzymes. Such complexes are particularly advantageous for hydrolysis of cellulosic biomass.

### BACKGROUND OF THE INVENTION

The plant cell wall is the most abundant renewable resource of biopolymer on earth. It is composed of various polysaccharides, mostly cellulose and hemicellulose, and lignin. Its degradation to soluble sugars is of great significance for conversion into desired chemicals and biofuels such as ethanol. Due to the highly ordered, insoluble, crystalline nature of the cellulose, very few microorganisms possess the necessary enzymatic system to efficiently degrade cellulosic substrates to soluble sugars.

Hydrolysis of cellulose is performed by a group of enzymes known as cellulases. They are classically divided into several groups: 1) exoglucanases, which can only cleave at the ends of the linear cellulose chain sequentially (2-4 glucose units at a time), and accordingly possess a tunnel-like active site; 2) endoglucanases, which cleave the cellulose chain in the middle (exposing new individual chain ends), commonly possess a groove, or cleft, which can fit any part of the linear chain; and 3) processive endoglucanases, considered as an intermediate group which, like endoglucanases, can cleave the cellulose chain in the middle but after the initial cleavage, can continue to sequentially degrade the cellulose chain like exoglucanases. Another classical group is β-glucosidases, which hydrolyze the terminal non-reducing β-D-glucose residues of cellodextrins (in particular cellobiose, which is one of the major end products of cellulose degradation) into monosaccharides.

Hemicellulose is degraded by a group of enzymes known as hemicellulases, that can be divided into two main types: those that cleave the main chain backbone (xylanases, which cleave randomly the β-1,4 linkage of xylan to produce xyloligosaccharides, which are further hydrolyzed into xylose by β-1,4 xylosidases); and those that degrade side chain substituents or short end products (such as arabinofuranosidase and acetyl esterases). Both type of enzymes (cellulases and hemicellulases) are needed in order to achieve complete plant cell wall degradation.

Plant cell wall-degrading microorganisms employ two major strategies: aerobic fungi and bacteria typically produce large amounts of free plant cell wall-degrading enzymes, whereas several anaerobic bacteria typically secrete a multi-enzymatic complex termed the cellulosome. The basic structure of a cellulosome complex includes a non-catalytic subunit called scaffoldin that binds the insoluble substrate via a cellulose-specific carbohydrate-binding module (CBM). The scaffoldin subunit also functions as an integrator of various enzymatic subunits into the complex—it typically contains a set of subunit-binding modules, termed cohesins, that mediate specific incorporation and organization of the enzymatic subunits into the complex through interaction with a complementary binding module, termed dockerin, that is present in each enzymatic subunit.

The cellulosome was first discovered in Clostridium thermocellum, which presents an elementary structure based on a primary scaffoldin molecule, which attaches to the substrate via a CBM and incorporates different enzymes via specific high-affinity cohesin-dockerin interactions. The cellulosome of C. thermocellum is incorporated into the cell surface via cohesin-dockerin interaction between the primary scaffoldin and an anchoring scaffoldin. The cohesin-dockerin partners that mediate the incorporation of the enzymes into the complex differ from those that mediate cell anchoring, such that there is essentially no cross-specificity between them, thus ensuring a reliable mechanism for cell-surface attachment and cellulosome assembly. The anchoring scaffoldin connects the complex to the cell via an SLH (S-layer homology) module (Bayer et al., 2004, Annual Review of Microbiology, 58: 21-554).

During the last two decades, the existence of more complex cellulosomal architectures were discovered, such as the cellulosomes in Acetivibrio cellulolyticus (Xu et al., 2003, J Bacteriol, 185(15): 4548-57; Xu et al., 2004, J Bacteriol, 186(17): 5782-9; Ding et al., 1999, J Bacteriol, 181(21): 6720-9) Ruminococcus flavefaciens (Saluzzi et al., 2001, FEMS Microbiol Ecol, 36(2-3): 131-137; Rincon et al., 2004, J Bacteriol, 186(9): 2576-85) Bacteroides Cellulosolvens (Xu et al., 2004, J Bacteriol, 186:968-977; Ding et al., 2000, J Bacteriol, 182:4915-4925) and more recently in Clostridium clariflavum (Izquierdo et al., 2012, Stand Genomic Sci, 6(1): 104-15).

The organization of the various scaffoldin modules into functional polypeptides is achieved by interconnecting linkers of different lengths and composition. The length of naturally occurring linkers shows great diversity, ranging from a few amino acids up to hundreds of amino acids. In some scaffoldins, neighboring cohesins may not be separated by linkers at all, such as the first and second or the third and fourth cohesins in ScaB from B. cellulosolvens (Bayer et al., 2009, Can we crystallize a cellulosome? In: Biotechnology of lignocellulose degradation and biomass utilization. Edited by Sakka K, Karita S, Kimura T, Sakka M, Matsui H, Miyake H, Tanaka A: Ito Print Publishing Division; 183-205). Molinier et al., 2011, J Mol Biol, 405:143-157, describe synergy, structure and conformational flexibility of hybrid cellulosomes containing scaffoldins composed of two cohesin modules, displaying various inter-cohesins linkers.

Designer cellulosomes are artificial nano-devices that allow controlled incorporation of plant cell wall degrading enzymes, and thus represent a potential platform for processing biomass to biofuels. It is based on the very high affinity and specific interaction between a cohesin and a dockerin module from the same species. Designer cellulosomes typically include a chimaeric scaffoldin containing a CBM and several cohesin modules derived from different species, having divergent specificities. The complex further includes plant cell wall-degrading enzymes, each having a complementary and specific dockerin module that mediates selective binding to one of the divergent cohesins.

Previous reports using designer scaffoldins resulted in enhanced activity of various recalcitrant substrates degradation (for example, Caspi et al., 2008, Journal of Biotechnology, 135: 351-357; Caspi et al., 2009, Applied and Environmental Microbiology, 75: 7335-7342; Morals et al., 2010, mBio, 1: e00285-10; Morals et al., 2011, mBio, 2: e00233-11). In most of these, configuration of designer cellulosomes mimicked the overall simple architecture of C. thermocellum. More complex structures are described, for example, in Mingardon et al., 2007, Applied and Environmental Microbiology, 73: 7138-7149.

One of the largest forms of homogeneous artificial cellulosome reported to date is described in Morals et al., 2012, MBio, 3(6), which contains a chimaeric scaffoldin with six divergent cohesins, integrating six dockerin-bearing cellulolytic enzymes (xylanases and cellulases).

Fan et al., 2012, PNAS U.S.A, 109(33): 13260-13265, describe the engineering of yeast to directly convert cellulose, especially microcrystalline cellulose, into bioethanol, through display of mini-cellulosomes composed of two individual mini-scaffoldins on the cell surface of Saccharomyces cerevisiae.

Tsai et al., 2013, ACS Synth. Biol., 2:14-21, describe functional display of complex cellulosomes on the yeast surface via adaptive assembly.

US 2011/0306105, to Chen et al., discloses designer cellulosomes for efficient hydrolysis of cellulosic material and more particularly for the generating of ethanol.

WO 2012/055863, to Fierobe et al., discloses covalent cellulosomes and uses thereof. In particular, enzyme constructs with increased enzymatic activity based on the use of spacers interconnecting catalytic modules are disclosed, and polynucleic acids encoding these constructs.

Vazana et al., 2013, Biotechnol Biofuels. 6(1):182, by some of the inventors of the present invention, published after the priority date of the present application, investigated the spatial organization of the scaffoldin subunit and its effect on cellulose hydrolysis by designing a combinatorial library of recombinant trivalent designer scaffoldins, which contain a carbohydrate-binding module (CBM) and three divergent cohesin modules.

There still remains a need for compositions and methods for improved degradation of cellulosic biomass. For example, it would be highly beneficial to have multi-enzyme complexes that allow the integration of a large number of cellulolytic enzymes working synergistically and effectively in order to achieve more efficient hydrolysis of cellulosic materials.

### SUMMARY OF THE INVENTION

The present invention provides artificial multi-enzyme complexes for efficient degradation of cellulosic biomass. More specifically, the present invention provides artificial multi-enzyme complexes comprising an array of scaffold subunits which allow the integration of an increased number of enzymes compared to previously described complexes, while maintaining efficient activity of each enzyme in the complex, and achieving overall synergy and proximity effects.

The present invention further provides compositions comprising the multi-enzyme complexes, and methods and systems for the hydrolysis of cellulosic material utilizing same.

The multi-enzyme complexes of the present invention comprise at least two scaffold subunits, where each subunit comprises a plurality of cohesin modules for integration of a plurality of carbohydrate active enzymes bearing matching dockerin modules. The cohesin modules of each subunit are separated by linkers of at least 5 amino acids, preferably 5-50 amino acids, which were found to result in improved activity of the complex, as exemplified herein below. The scaffold subunits also interact with each other, via cohesin-dockerin interaction with a binding specificity that is different from the binding specificities that connect each scaffold and its enzymes, thereby generating an elaborate structure incorporating a large number of enzymes.

Advantageously, the precise position of each enzyme in the complex can be controlled, by using scaffolds comprising cohesin modules of different specificities, that can interact with their matching dockerins modules on the enzymes.

The number of cohesin-dockerin pairs with divergent binding specificities is limited. The use of a structure composed of multiple scaffold subunits as described herein overcomes this limitation: cohesin modules of the same specificity can be used on different scaffolds. Each scaffold can be separately interacted with its enzymes before the scaffolds themselves are reacted to form the entire complex. Once the individual complexes are formed they are stable, thus, the specific position of each enzyme is maintained. The multi-enzyme complexes disclosed herein permit higher flexibility in the selection of cohesin modules and control of enzyme composition and assembly. The resulting complexes incorporate multiple enzymes in a configuration that allows optimal activity and synergism.

According to one aspect, the present invention provides an artificial cellulolytic multi-enzyme complex comprising:

a first scaffold polypeptide comprising a plurality of cohesin modules separated by linkers comprising 5-50 amino acids, at least two of said cohesin modules having distinct binding specificities for dockerin modules, and a dockerin module;

a second scaffold polypeptide comprising a plurality of cohesin modules separated by linkers comprising 5-50 amino acids, at least two of said cohesin modules having distinct binding specificities for dockerin modules, wherein at least one of the cohesin modules has binding specificity for the dockerin of the first scaffold polypeptide; and

a plurality of carbohydrate active enzymes, each carbohydrate active enzyme comprises a dockerin module with a binding specificity for a cohesin of the first scaffold, second scaffold or both,

wherein the first and second scaffolds are bound via the dockerin of the first scaffold and the cohesin of the second scaffold having a binding specificity for said dockerin, and

wherein the plurality of carbohydrate active enzymes are bound to the first and second scaffold polypeptides via dockerin-cohesin modules having mutual binding specificities, and

wherein the first scaffold, second scaffold or both further comprise a carbohydrate binding module (CBM).

As used herein, the term “distinct specificity”, when referring to a binding specificity of cohesin modules, is used interchangeably with “divergent specificity” and indicates that each cohesin module recognizes a different dockerin module. In some embodiments, cohesin modules of distinct binding specificities originate from different microorganism species. According to these embodiments, cohesin modules originating from one species recognize (bind) dockerin modules originating from the same species but not dockerin modules originating from a different species.

Similarly, when the terms “distinct specificity” and “divergent specificity” refer to a binding specificity of dockerin modules, they indicate that each dockerin module recognizes a different cohesin module.

As used herein, the term “mutual”, when referring to a dockerin-cohesin interaction, indicates that the two modules are complementary to each other, namely, having binding specificity for each other.

In some embodiments, each of the first and second scaffold polypeptides comprises 3-10 cohesin modules. In some embodiments, each of the first and second scaffold polypeptides comprises 3-6 cohesin modules.

In some embodiments, all cohesin modules of the first scaffold polypeptide are of distinct binding specificities. In additional embodiments, all cohesin modules of the second scaffold polypeptide are of distinct binding specificities. According to these embodiments, the first scaffold has a set of divergent cohesin modules, and the second scaffold has another set of divergent cohesin modules. In some embodiments, all cohesins, in both sets, differ from each other. In other embodiments, each set includes divergent cohesins, but one (or more) cohesins may be found in both sets. Thus, in some embodiments, at least one of the cohesin modules of the first scaffold has the same binding specificity as a cohesin module of the second scaffold. As noted above, the position of the enzymes can still be maintained within each scaffold by forming each scaffold-enzyme complex separately, and then mixing the pre-formed complexes to generate the entire complex.

In other embodiments, the first scaffold polypeptide or the second scaffold polypeptide comprises two or more cohesin modules with the same binding specificity. In some embodiments, both scaffold polypeptides comprise two or more cohesin modules of the same specificity, i.e., each scaffold polypeptide comprises two or more cohesin modules with the same binding specificity. Such embodiments may be useful, for example, for the integration of a particular enzyme in multiple positions within the complex.

In some embodiments, the cohesin modules originate from one or more cellulosome-producing microorganisms. In some embodiments, the cellulosome-producing microorganisms are selected from the group consisting of Clostridium thermocellum, Acetivibrio cellulolyticus, Ruminococcus flavefaciens, Bacteroides cellulosolvens, Archaeoglobus fulgidus and Clostridium cellulolyticum. According to these embodiments, the cohesin modules are selected from the group consisting of cohesins from C. thermocellum, cohesins from A. cellulolyticus, cohesins from R. flavefaciens, cohesins from B. cellulosolvens, cohesins from A. fulgidus, cohesins from C. cellulolyticum and combinations thereof.

In some embodiments, the cohesin modules originate from one or more non-cellulosomal microorganisms.

In some embodiments, the dockerin modules originate from one or more cellulosome-producing microorganisms. In some embodiments, the cellulosome-producing microorganisms are selected from the group consisting of C. thermocellum, A. cellulolyticus, R. flavefaciens, B. cellulosolvens, A. fulgidus and C. cellulolyticum. According to these embodiments, the dockerin modules are selected from the group consisting of dockerins from C. thermocellum, dockerins from A. cellulolyticus, dockerins from R. flavefaciens, B. cellulosolvens, dockerins from A. fulgidus, dockerins from C. cellulolyticum and combinations thereof.

In some embodiments, the dockerin modules originate from one or more non-cellulosomal microorganisms.

In some embodiments, both first and second scaffold polypeptides comprise a CBM.

In some embodiments, the CBM of the first scaffold polypeptide, the second scaffold polypeptide or both is internal. In some embodiments, the CBM of the first scaffold polypeptide, the second scaffold polypeptide or both is positioned at a terminus of the scaffold polypeptide.

In some embodiments, when both scaffold polypeptides comprise a CBM, the CBM of the first and second scaffold polypeptide are the same. In other embodiments, the CBM of the first and second scaffold polypeptide are different.

In some embodiments, the linkers are composed of 5-40 amino acids. In some embodiments, the linkers are composed of 15-35 amino acids.

In some embodiments, the plurality of carbohydrate active enzymes comprises glycoside hydrolases, polysaccharide lyases, carbohydrate esterases or combinations thereof.

In some embodiments, the glycoside hydrolases are selected from the group consisting of cellulases, xylanases, β-glucosidases and combinations thereof.

In some embodiments, the carbohydrate-active enzymes originate from non-cellulosomal enzymes.

In some embodiments, the carbohydrate-active enzymes originate from cellulosomal enzymes.

In some embodiments, the carbohydrate-active enzymes are bacterial enzymes. In some embodiments, the bacteria are selected from the group consisting of Thermobifida fusca and Clostridium thermocellum. According to these embodiments, the multi-enzyme complex comprises a plurality of carbohydrate-active enzymes from T. fusca, C. thermocellum or both.

In some embodiments, the multi-enzyme complex further comprises one or more scaffold polypeptides with a plurality of carbohydrate binding enzymes bound thereto, bound to the first scaffold polypeptide, second scaffold polypeptide or both.

According to another aspect, the present invention provides a composition for degrading a cellulosic material comprising the multi-enzyme complex of the present invention.

According to yet another aspect, the present invention provides a system for degrading a cellulosic material, the system comprising the multi enzyme complex of the present invention.

According to yet another aspect, the present invention provides a method for degrading a cellulosic material, the method comprising exposing said cellulosic material to the multi-enzyme complex of the present invention.

These and further aspects and features of the present invention will become apparent from the figures, detailed description, examples and claims which follow.

### BRIEF DESCRIPTION OF THE FIGURES

FIG. 1. Schematic representation of a scaffold library constructed to examine the effect of the length of inter-module linkers on activity of a scaffold-enzyme complex. Twenty-four (24) different arrangements of cohesin modules (Ac, Bc and Ct) and a carbohydrate binding module (CBM) are shown in three sub-libraries: no-linker, short-linker and long-linker versions of the given chimaeric scaffold. The left columns indicate the number of each scaffold set and its composition (position of CBM and divergent cohesins). Fourteen (14) full sets, representing 42 cloned and expressed scaffolds, were successfully cloned and expressed and used for further study. The 42 successfully cloned and expressed scaffoldins included in the final library are shown as grayscale pictograms.

FIG. 2. Comparative hydrolysis of Avicel (A) and pretreated cellulose-enriched wheat straw (B) by 14 sets of designer cellulosomes. The modular composition of each set and the scaffoldin number is denoted on the x-axis. Upper panel: the CBM module of the designer scaffoldin is in an internal position. Lower panel: the CBM module of the designer scaffoldin is at the N- or C-terminal position. Each designer-cellulosome set is assembled with either long intermodular linker scaffoldin, short intermodular linker scaffoldin and no intermodular linker scaffoldin. Controls: “Free”: corresponds to the combined activity of Cel48S-ct, Cel9K-ac and Cel8A-bc. “CBM-Coh”: corresponds to the activity of the former three enzymes, each attached separately to its matching cohesin module fused to a CBM. Reactions were carried out for 72 h on Avicel and for 3 h on pretreated cellulose-enriched wheat straw. Enzymatic activity was defined by mM reducing sugars as determined by a glucose standard curve. All reactions were carried out in triplicate and repeated three times. Standard deviations of at least three experiments are indicated.

FIG. 3. Activity assay on Avicel comparing a-9A, b-48A and 5A-t as: (i) bound to the adaptor scaffold CBM-cohesins A-B-T-DockII (“Scad ABT”); (ii) bound to the adaptor scaffold DockII-A-B-T that is further bound to a matching cohesin-CBM mini-scaffold (“Ad ABT”); (iii) mixture of free enzymes (“Free”); and (iv) mixture of enzymes bound to matching cohesin-CBM mini-scaffolds (“CBM-restored”).

FIG. 4. A schematic illustration of a multi-enzyme complex containing a hexavalent primary scaffold, a trivalent adaptor scaffold, and eight enzymatic subunits.

FIG. 5. Wheat straw degradation after 48 hours incubation at 50° C. with different chimaeric enzymatic cocktails and cellulosomal configurations. Presence of the various components in each reaction solution is specified in the table.

FIG. 6. Kinetics of wheat straw degradation (50° C.) by: (i) extracted natural cellulosome of C. thermocellum; (ii) a designer cellulosome containing an adaptor scaffold attached to a hexavalent scaffold with a total of eight chimaeric enzymes; and (iii) mixture of the corresponding eight wild-type enzymes; in the presence or absence of a betaglucosidase (BglC from T. fusca).

### DETAILED DESCRIPTION OF THE INVENTION

The present invention is directed to designer cellulosomes having elaborate structure composed of two (and possibly more) interacting scaffold subunits. The scaffold subunits of the present invention are designed such that they allow efficient integration of enzymatic subunits to the complex, and promote proximity and targeting effects for efficient degradation of cellulosic substrates.

In some embodiments, there is provided herein an artificial cellulolytic multi-enzyme complex comprising: (i) a first plurality of carbohydrate active enzymes, each comprising a dockerin module, bound to a first scaffold polypeptide, wherein said first scaffold polypeptide comprises a plurality of cohesin modules separated by linkers comprising 5-50 amino acids and having binding specificities for the dockerin modules of the enzymes, a carbohydrate binding module (CBM), and a dockerin module; (ii) a second plurality of carbohydrate active enzymes, each comprising a dockerin module, bound to a second scaffold polypeptide, wherein said second scaffold polypeptide comprises a plurality of cohesin modules separated by linkers comprising 5-50 amino acids, wherein at least one of the cohesin modules has binding specificity for the dockerin of the first scaffold, and the remaining cohesin modules have binding specificities for the dockerin modules of the second plurality of enzymes, wherein the first and second scaffolds are bound via the dockerin of the first scaffold and the cohesin of the second scaffold having binding specificity for said dockerin.

As used herein, the term “artificial”, when referring to the enzymatic complex of the present invention, indicates that the complex is made artificially/synthetically and does not occur in nature. It is to be understood that naturally occurring cellulosome complexes are excluded from the scope of the present invention.

As used herein, the term “enzyme” refers to a polypeptide having a catalytic activity towards a certain substrate or substrates.

As used herein, the term “module” describes a separately folding moiety within a protein. The “catalytic module of an enzyme” or “an enzymatically-active module”, as used herein, refers to a module which contributes the catalytic activity to a protein. The terms refer to their accepted interpretation for modular enzymes, for which the catalytic module can be readily identified within the enzyme polypeptide sequence. Such modular enzymes are under the scope of the present invention.

The term “complex” as used herein refers to a coordination or association of components linked preferably by non-covalent interactions, or by covalent bonds.

The term “multi-enzyme complex” as used herein indicates a complex comprising a plurality of enzymes, namely, at least two enzymes and preferably more. The multi-enzyme complex of the present invention further includes non-catalytic components, such as structural components and substrate-binding components.

As used herein, the term “plurality” indicates at least two.

As used herein, the term “scaffold polypeptide” or a “scaffold subunit” are used interchangeably and refer to a backbone subunit that provides a plurality of binding sites for enzymatic and/or non-enzymatic protein components. Thus, the scaffold polypeptide serves as a platform for integration of components, both enzymes and non-enzymatic protein components. The scaffold polypeptide is typically non-catalytic. The scaffold polypeptide may include one or more substrate-binding modules.

As used herein, the term “carbohydrate active enzyme” refers to an enzyme that catalyzes the breakdown of carbohydrates and glycoconjugates. The term encompasses enzymatically-active portions of enzymes that catalyze the breakdown of carbohydrates and glycoconjugates. The broad group of carbohydrate active enzymes is divided into enzyme classes and further into enzyme families according to a standard classification system (Cantarel et al. 2009 Nucleic Acids Res 37:D233-238). According to this classification system, three classes of enzymes that involve in the breakdown of carbohydrates and glycoconjugates are defined, namely (i) glycoside hydrolases, which hydrolyze glycosidic bonds between two or more carbohydrates or between a carbohydrate and a non-carbohydrate moiety, including for example, cellulases, xylanase, α-L-arabinofuranosidase, cellobiohydrolase, β-glucosidase, β-xylosidase, β-mannosidase and mannanase; (ii) polysaccharide lyases, which catalyze the breakage of a carbon-oxygen bond in polysaccharides leading to an unsaturated product and the elimination of an alcohol, for example, pectate lyases and alginate lyases; and (ii) carbohydrate esterases, which catalyze the de-O or de-N-acylation of substituted saccharides, for example, acetylxylan esterases, pectin methyl esterases, pectin acetyl esterases and ferulic acid esterases. An informative and updated classification of carbohydrate active enzymes is available on the Carbohydrate-Active Enzymes (CAZy) server (www.cazy.org).

Along with the classification system, a unifying scheme for designating the different catalytic modules and the different carbohydrate active enzymes was suggested and has been widely adopted. A catalytic module is designated by its enzyme class and family number. For example, a glycoside hydrolase having a catalytic module classified in family 10 is designated as “GH10”. An enzyme is designated by the type of activity, the family it belongs to and typically an additional letter. For example, a cellulase from a certain organism having a catalytic module classified as family 5 glycoside hydrolase, which is the first reported GH5 cellulase from this organism, is designated as “Cel5A”.

The terms “polypeptide,” “peptide” and “protein” are used interchangeably herein to refer to a polymer of amino acid residues.

The terms “polynucleotide” or “oligonucleotide” are used interchangeably herein to refer to a polymer of nucleic acids.

As used herein, the term “wild type” refers to the naturally occurring DNA/protein. The terms “derivative”, “variant”, “modified” are used interchangeably and refer to a polypeptide which differs from a wild-type amino acid sequence due to one or more amino acid substitutions introduced into the sequence, and/or one or more deletions/additions. It is to be understood that a derivative/variant generally retains the properties or activity observed in the wild-type to the extent that the derivative is useful for similar purposes as the wild-type form. For example, when the terms refer to a cohesin or dockerin, they indicate that the wild-type sequence has been modified without adversely affecting its ability to recognize the matching cohesin/dockerin, respectively. Typically, the recognition site of the relevant counterpart, also referred to as the binding site, is maintained. When referring to an enzyme, the terms indicate that the wild-type sequence has been modified without adversely affecting its catalytic activity. Typically, the catalytic domain is maintained.

Multi-Enzyme Complexes

Cohesins and Dockerins

The assembly of the multi-enzyme complex according to embodiments of the present invention is mediated by a protein-protein interaction between two modules—cohesins and dockerins.

In natural cellulosome systems, cohesin and dockerin modules govern the integration of enzymes into a scaffoldin subunit, as well as the attachment of the cellulosome to the surface of a cellulosome-producing microorganism (in some cellulosome-producing microorganisms).

The cohesins are modules of approximately 140 amino acid residues, that typically appear as repeats as part of the structural scaffoldin subunit. There are three major types of cohesin modules, types I, II and III, which are classified based on amino acid sequence homology and protein topology. Classification of a given cohesin can be carried out through sequence alignment to known cohesin sequences. The sequence of type-II cohesin domains are characterized by two insertions which are not found in type-I cohesin domains. Topologically, all cohesin types share a common structure of nine-stranded β-sandwich with jellyroll topology. Type I cohesin includes only the basic jellyroll structure. The structure of the type-II cohesin module has an overall fold similar to that of type-I, but includes distinctive additions: two β-flaps' interrupting strands 4 and 8 and an α-helix at the crown of the protein module. The structure of the type-III cohesin module is similar to that of type-II, namely, it includes two β-flaps' interrupting strands 4 and 8 and an α-helix, but the location of the α-helix differs from that of type-II. In addition, type-III is characterized by an extensive N-terminal loop.

The dockerins are modules of approximately 60-70 amino acid residues, characterized by two duplicated c. 22-residue segments, frequently separated by a linker of 9-18 residues. The two repeats include a calcium-binding loop and an T-helix′ motif. The dockerins are classified into types according to the cohesin with which they interact, and similarly include types I, II and III. The phylogenetic map of the dockerins reflects, to a great extent, that of their cohesin counterparts, such that dockerins that interact with type-I cohesins are closely grouped, and the dockerins that interact with the type-II cohesins are also grouped and distant from the first group.

Interactions among type-I modules generally observe cross-species stringency of the cohesin-dockerin system, such that type-I cohesin of one microorganism species would not be expected to recognize type-I dockerins from a different microorganism species. Within a given species, however, type-I interactions tend to be non-specific, such that all cohesins on a primary scaffoldin tend to bind similarly to different enzyme-borne dockerins. Thus, within a given species, cohesin modules that serve for enzyme incorporation generally have similar specificities. Inter-species specificity of interactions among type-II modules appears to be much less strict than that observed for type-I, and cross-species interaction is sometimes observed. There is essentially no cross-specificity between type I and type II cohesin-dockerin partners.

The cohesin modules constitute the scaffold subunits. Dockerin modules with corresponding binding specificity are selected for the enzymes to be integrated into the complex. For the construction of a scaffold subunit that integrates enzymes to precise locations, cohesins of divergent specificities should be selected. For example, each cohesin can originate from a different microorganism. As another example, cohesins from the same species but of different types can be selected.

Information about classification of cohesin and dockerin modules can be found, for example, in Albar et al. (2009) Proteins, 77:699-709; Noach et al. (2005) J. Mol. Biol. 348, 1-12, Xu et al. (2003) J. Bacteriol. 185: 4548-4557; Bayer et al. (2004) Annu. Rev. Microbiol. 58:521-54; Peer et al. (2009) FEMS Microbiol Lett., 291(1): 1-16.

Information about inter- and intra-species specificity among type I and type II cohesins and dockerins may be found, for example, in Haimovitz et al. (2008) Proteomics, 8, 968-979.

Non-limiting examples of cohesin-dockerin pairs with mutual binding specificities that can be used for the construction of multi-enzyme complexes according to embodiments of the present invention are specified in Table A below:

TABLE A
cohesin-dockerin pairs

| | Cohesin | | Dockerin | |
|---|---|---|---|---|
| | | Accession No./ | | Accession No./ |
| Origin | Name | sequence | Name | sequence |
| C. thermocellum | cohesin of CipA | SEQ ID NO: 2 | dockerin of | Residues 652-715 |
| | (e.g., second or | | Cel48S | of |
| | third cohesin) | | | SEQ ID NO: 13 |
| | | | dockerin of | Residues 313-376 |
| | | | Xyn10Z | of |
| | | | | SEQ ID NO: 37 |
| B. cellulosolvens | cohesin of ScaB | SEQ ID NO: 3 | dockerin of | Residues 389-459 |
| | (e.g., third | | ScaA | of |
| | cohesin) | | | SEQ ID NO: 15 |
| A. cellulolyticus | cohesin of ScaC | SEQ ID NO: 4 | dockerin | Residues 808-878 |
| | (e.g., third | | module of | of |
| | cohesin) | | ScaB | SEQ ID NO: 17 |
| C. thermocellum | type II cohesin | UniProtKB | type II | GenBank |
| | module from a | Q06853 | dockerin | ABN54273 or |
| | cell surface- | P71143, | module | UniProtKB/ |
| | anchoring | Q06852 (SEQ | from CipA | Swiss-Prot: |
| | protein: Orf2p, | ID NO: 42), | | Q46453 |
| | SdbA, OlpB, | A3DDE1, | | |
| | Cthe_0735 and | A3DDE2 | | |
| | Cthe_0736 | | | |
| C. cellulolyticum | cohesin from | SEQ ID NO: | dockerin | Residues 564-623 |
| | scaffoldin C | 39 | from | of |
| | (e.g., cohesin | | scaffoldin A | SEQ ID NO: 45 |
| | 1) | | | |
| R. flavefaciens | cohesin from | SEQ ID NO: | dockerin | Residues 368-444 |
| | scaffoldin B of | 41 | from ScaA | of |
| | strain 17 (e.g., | | | SEQ ID NO: 53 |
| | cohesin 1) | | | |
| A. fulgidus | cohesin 2375 | SEQ ID NO: | dockerin | Residues 431-506 |
| | | 40 | 2375 | of |
| | | | | SEQ ID NO: 51 |

Examples of additional cohesin-dockerin pairs are available in the scientific literature and are known to persons of skill in the art.

Interacting cohesin and dockerin pairs can be taken from natural cellulosome-producing bacteria, for example, from scaffoldins and/or enzymes found in C. thermocellum, C. cellulolyticum, C. cellulovorans, C. josui, C. papyrosolvens, C. clariflavum, B. cellulosolvens, A. cellulolyticus.

Interacting cohesin and dockerin pairs can also be taken from non-cellulosomal bacteria and archaea. Non-cellulosomal cohesin-dockerin interaction was first described in Bayer et al., 1999, FEBS Lett. 463: 277-280. A non-limiting list of such non-cellulosomal cohesin and dockerin modules can be found in the supporting information of Peer et al., 2009, FEMS Microbiol Lett. 291: 1-16.

In some embodiments, the scaffold polypeptides of the present invention include 2-10 cohesin modules, for example 2-8 cohesin modules, for example 3-8, for example 3-6. In some embodiments, an adaptor scaffold (first scaffold) that integrates enzymes and attaches to a primary scaffold (second scaffold) comprises 3-4 cohesin modules. An adaptor scaffold typically further comprises a dockerin module for attachment to a cohesin on a primary scaffold. In some embodiments, a primary scaffold polypeptide, which integrates enzymes and/or adaptor scaffold(s) comprises 4-6 cohesin modules. The binding specificity between the scaffolds is different from the binding specificity of the scaffolds and enzymes.

In some embodiments, an adaptor scaffold comprises a plurality of cohesin modules, wherein at least two of the cohesin modules have distinct binding specificities for dockerin modules. According to these embodiments, the adaptor scaffold comprises two divergent cohesin modules, each recognizes a different dockerin. Further cohesin modules that may be present in the adaptor scaffold may have distinct or the same binding specificity. In some embodiments, all cohesin modules of the adaptor scaffold have distinct binding specificities, meaning that each cohesin on the adaptor scaffold recognizes a different dockerin.

Primary scaffolds of the present invention comprise a plurality of cohesin modules, wherein at least one of the cohesin modules has binding specificity for the dockerin of an adaptor scaffold.

In some typical embodiments, a primary scaffold of the present invention further comprises one or more cohesin modules for integration of enzymes. Those cohesin modules are typically characterized by binding specificities that are different from that of the cohesin module that serves to bind an adaptor scaffold. In some embodiments, the cohesin modules for enzyme integration have distinct binding specificities, such that each cohesin recognizes a different dockerin.

In some embodiment, a primary scaffold comprises a plurality of cohesin modules, wherein the plurality of cohesin modules comprises a cohesin module having a binding specificity for the dockerin of an adaptor scaffold, and a cohesin module with a binding specificity for a dockerin other than the dockerin of the adaptor scaffold.

In some embodiments, at least one of the cohesin modules of the adaptor scaffold has the same binding specificity as a cohesin module of the primary scaffold, meaning that at least one cohesin module of a particular binding specificity is found on both the primary and adaptor scaffolds.

In some embodiments, the scaffold polypeptides of the present invention further comprise one or more carbohydrate binding modules (CBM). In some embodiments, the CBM is a cellulose-binding CBM. In other embodiments, the CBM is a xylan-binding CBM. In some embodiments, the CBM is classified in a CBM family selected from the group consisting of family 1, 2 and 3, as defined in the CAZY server and/or CAZYpedia as detailed above. In some embodiments, the CBM originates from C. thermocellum CBMs. In some exemplary embodiments, the C. thermocellum CBM is CBM3a of the scaffoldin subunit CipA (GenBank Accession No. ABN54273).

In some embodiments, the multi-enzyme complexes of the present invention comprise an array of primary and adaptor scaffolds for integration of the enzymes, where the adaptor scaffold is an intermediate scaffold that incorporates various enzymes and also attaches to the primary scaffold.

In some embodiments, a multi-enzyme complex is provided, containing: a primary scaffold, a first set of enzymes bound to the primary scaffold, and an adaptor scaffold with a second set of enzymes, the adaptor scaffold is bound to the primary scaffold.

In some exemplary embodiments, a first (adaptor) scaffold polypeptide of the present invention comprises a type II dockerin from C. thermocellum, a cohesin from A. cellulolyticus, a cohesin from B. cellulosolvens, a cohesin from C. thermocellum and a CBM from C. thermocellum. In some embodiments, these modules are separated by linkers of 15-40 amino acids, for example 25-40 amino acids.

In some exemplary embodiments, a second (primary) scaffold polypeptide of the present invention comprises a cohesin from C. cellulolyticum, a cohesin from A. cellulolyticus, a type I cohesin from C. thermocellum, a cohesin from A. fulgidus, a cohesin from R. flavefaciens, a type II cohesin from C. thermocellum and a CBM from C. thermocellum. In some embodiments, these modules are separated by linkers of 15-40 amino acids, for example 25-40 amino acids.

In some embodiments, the adaptor scaffold comprises a sequence having at least 80% identity with the sequence set forth in SEQ ID NO: 31, for example, at least 85%, at least 90%, at least 95%, at least 97% identity with the sequence set forth in SEQ ID NO: 31. In some exemplary embodiments, the adaptor scaffold comprises the sequence set forth in SEQ ID NO: 31.

In some embodiments, the primary scaffold comprises a sequence having at least 80% identity with the sequence set forth in SEQ ID NO: 43, for example, at least 85%, at least 90%, at least 95%, at least 97% identity with the sequence set forth in SEQ ID NO: 43. In some exemplary embodiments, the primary scaffold comprises the sequence set forth in SEQ ID NO: 43.

It is to be understood that changes introduced into the sequences set forth in SEQ ID NOs. 31 and 43 should not be made in the regions corresponding to binding sites of cohesins with their respective dockerins, which are important for this interaction.

Linkers

The different modules of the scaffold polypeptides of the present invention are interconnected by linkers composed of 5 amino acids or more, typically of 5-50 amino acids, for example 5-35 amino acids, 15-50 amino acids, 20-50 amino acids, 25-50 amino acids, 20-40 amino acids, 25-45 amino acids, 25-40, 15-35 amino acids. Each possibility represents a separate embodiment of the present invention.

In some embodiments, the linkers interconnecting modules of a particular scaffold polypeptide are the same. In some embodiments, different linkers are used within one scaffold polypeptide, between the different components.

Linker regions are generally composed of a restricted set of amino acids—typically prolines and threonines are prevalent with additional types of amino acids less abundant.

The composition of amino acids for the linkers can be selected, for example, to include the sequence of a linkers (or a portion thereof) adjacent to the modules (i.e., cohesins, CBM, etc) used to fabricate the chimaeric scaffold subunit. Sequences of linkers for the construction of the scaffold polypeptides of the present invention can be derived, for example, from the list reviewed in Bayer et al., 2009, Can we crystallize a cellulosome? In: Biotechnology of lignocellulose degradation and biomass utilization. Edited by Sakka K, Karita S, Kimura T, Sakka M, Matsui H, Miyake H, Tanaka A: Ito Print Publishing Division; 183-205). Exemplary linker sequences are provided in the Examples section below.

Enzymes

The scaffold polypeptides of the present invention mediate, according to some embodiments, the integration of a plurality of carbohydrate active enzymes or enzymatically-active portions thereof into the complex. Each enzyme, or an enzymatically-active portion thereof, comprises a dockerin module for integration into a specific matching cohesin.

In some embodiments, an enzyme integrated into the complex comprises a heterologous dockerin module. A heterologous dockerin module indicates either a dockerin that is different from the naturally-occurring dockerin of the enzyme, or a dockerin that is introduced into a polypeptide that does not naturally include a dockerin, i.e., it is an engineered enzyme derived from a wild-type sequence that does not include a dockerin module. The wild-type is therefore unable to incorporate into complexes such as the cellulosome. The engineered enzyme, however, is designed to include a dockerin module and is therefore capable of integrating into the complex of the present invention.

Typically, carbohydrate active enzymes are characterized by a multi-modular organization, where the catalytic module is associated with one or more ancillary, helper, modules which modulate the enzyme activity. Each module comprises a consecutive portion of the polypeptide chain and forms an independently folding, structurally and functionally distinct unit. One of the main ancillary modules is the carbohydrate-binding module. In some embodiments, the heterologous dockerin domain replaces at least one ancillary module originally found in the enzyme structure. In other embodiments, the heterologous dockerin domain is introduced in addition to the original ancillary modules.

In some embodiments, the carbohydrate active enzymes are selected from the group consisting of glycoside hydrolases, polysaccharide lyases and carbohydrate esterases. In some embodiments, combinations of glycoside hydrolases, polysaccharide lyases and carbohydrate esterases are used.

As noted above “glycoside hydrolases” are enzymes that hydrolyze glycosidic bonds between two or more carbohydrates or between a carbohydrate and a non-carbohydrate moiety. The glycoside hydrolases may catalyze the hydrolysis of O-, N- and/or S-linked glycosides. The glycoside hydrolases are sometimes referred to as glycosidases and glycosyl hydrolases. Non-limiting examples of glycoside hydrolases include a cellulase, xylanase, α-Larabinofuranosidase, cellobiohydrolase, β-glucosidase, β-xylosidase, β-mannosidase and mannanase. Information about glycosidic bonds and other types of bonds found in carbohydrate molecules, can be found, for example, in M. L. Sinnott (2007) Carbohydrate Chemistry and Biochemistry: Structure and mechanism, 1st edition, Royal Society of Chemistry.

In some particular embodiments, the glycoside hydrolases of the complex of the present invention are selected from the group consisting of cellulases, xylanases and β-glucosidases. In some embodiments, combinations of cellulases, xylanases and β-glucosidases are used.

As further noted above, “polysaccharide lyases” refers to a group of carbonoxygen lyases that catalyze the breakage of a carbon-oxygen bond in polysaccharides leading to an unsaturated product and the elimination of an alcohol. Typically, polysaccharide lyases cleave uronic acid-containing polysaccharide chains via a β-elimination mechanism, to generate an unsaturated hexenuronic acid residue and a new reducing end. Non-limiting examples of polysaccharide lyases include pectate lyase and alginate lyase.

As further noted above, “carbohydrate esterases” refers to enzymes that hydrolyze carbohydrate esters. Typically, carbohydrate esterases catalyze the de-O or de-N-acylation of substituted saccharides. Non-limiting examples of carbohydrate esterases include acetylxylan esterase, pectin methyl esterase, pectin acetyl esterase and ferulic acid esterases.

In some embodiments, the carbohydrate-active enzymes are cellulosomal enzymes. The term “cellulosomal enzyme” refers to an enzyme that in nature is typically found as part of a cellulosome complex.

In some embodiments, the carbohydrate-active enzymes are non-cellulosomal enzymes. The term “non-cellulosomal enzyme” refers to an enzyme that in nature is active as a free enzyme, typically secreted into the environment. Such enzymes usually do not have a dockerin module.

In some embodiments, the carbohydrate-active enzymes are bacterial enzymes. In some embodiments, the bacteria are selected from the group consisting of T. fusca and C. thermocellum. In other embodiments, the carbohydrate-active enzymes are fungal enzymes.

Types of carbohydrate active enzymes are described above. In some embodiments, the carbohydrate active enzymes include xylanases. Xylanases are classified, for example, in glycoside hydrolase families 5, 8, 10, 11, 26 and 43. In some embodiments, the xylanases are bacterial xylanases.

In some embodiments, the carbohydrate active enzymes include cellulases. The cellulases may be selected from exoglucanases, endoglucanases and proccessive-endoglucanase. Cellulases are classified, for example, in glycoside hydrolase families 5, 6, 7, 8, 9, 12, 26, 44, 45, 48, 51, 61, and 74. In some embodiments, the cellulases are bacterial cellulases.

In some embodiments, the carbohydrate active enzymes include β-glucosidases. β-glucosidases are classified, for example, in glycoside hydrolase families 1, 3, 9, 30 and 116. In some embodiments, the β-glucosidases are bacterial β-glucosidases.

In some exemplary embodiments, a plurality of carbohydrate active enzymes bound to a scaffold polypeptide comprises an exoglucanase, an endoglucanase, and a processive-endoglucanase.

In some embodiments, a multi-enzyme complex of the present invention comprises at least two cellulases, for example three cellulases, four cellulases, or more. Each possibility represents a separate embodiment of the present invention.

In some embodiments, a multi-enzyme complex of the present invention comprises at least two xylanases, for example xylanases cellulases, xylanases cellulases, or more. Each possibility represents a separate embodiment of the present invention.

In some exemplary embodiments, a multi-enzyme complex of the present invention comprises four cellulases and four xylanases.

In some specific exemplary embodiments, the plurality of carbohydrate active enzymes comprises at least one of the exoglucanase Cel48S from C. thermocellum, the endoglucanase Cel8A from C. thermocellum and the proccessive-endoglucanase Cel9K from C. thermocellum.

In additional specific exemplary embodiments, the plurality of carbohydrate active enzymes comprises at least one of the exoglucanase Cel48A from T. fusca, the endoglucanase Cel5A from T. fusca, and the proccessive-endoglucanase Cel9A from T. fusca.

In additional specific exemplary embodiments, the plurality of carbohydrate active enzymes comprises at least one of the xylanses Xyn43A, Xyn11A, Xyn10B, and Xyn10A from T. fusca.

In some exemplary embodiments, a plurality of carbohydrate active enzymes bound to a scaffold polypeptide comprises xylanases and an exoglucanase.

In some specific exemplary embodiments, the plurality of carbohydrate active enzymes comprises the xylanses Xyn43A, Xyn11A, Xyn10B, and Xyn10A from T. fusca, and the exoglucanase Cel5A from T. fusca.

Exemplary enzymatic subunits with suitable dockerins are provided in the Examples section below.

For some combinations of enzymes, the arrangement, or relative order, within the complex has an effect on the overall activity. The effect of the arrangement of the activity of the complex can be readily determined by a person skilled in the art.

In some typical embodiments, the scaffold polypeptides and each of the carbohydrate active enzymes present in the multi-enzyme complexes of the present invention are non-covalently linked. In additional typical embodiments, they are linked via an interaction between the cohesins and dockerins. In other embodiments, the scaffold polypeptides and each of the cellulolytic enzymes are covalently linked. In additional or alternative embodiments, the scaffold polypeptide and each of the cellulolytic enzymes are crosslinked.

Typically, the different components of the multi-enzyme complex are produced recombinantly and separately in host cells, purified, and then mixed together in a solution to form the complex.

Thus, the multi-enzyme complex is typically unattached to the outer surface of a microorganism cell.

The polypeptides described herein may be produced by recombinant methods, as know in the art. For example:

Recombinant Expression

The polypeptides of the present invention may be synthesized by expressing a polynucleotide molecule encoding the polypeptide in a host cell, for example, a microorganism cell transformed with the nucleic acid molecule.

The synthesis of a polynucleotide encoding the desired polypeptide may be performed as described in the Examples below. DNA sequences encoding wild type polypeptides may be isolated from any strain or subtype of a microorganism producing them, using various methods well known in the art (see for example, Sambrook, et al., Molecular Cloning: A Laboratory Manual, Third Edition, Cold Spring Harbor, N.Y., (2001)). For example, a DNA encoding the wild type polypeptide may be amplified from genomic DNA of the appropriate microorganism by polymerase chain reaction (PCR) using specific primers, constructed on the basis of the nucleotide sequence of the known wild type sequence. The genomic DNA may be extracted from the bacterial cell prior to the amplification using various methods known in the art, see for example, Marek P. M et al., “Cloning and expression in Escherichia coli of Clostridium thermocellum DNA encoding p-glucosidase activity”, Enzyme and Microbial Technology Volume 9, Issue 8, August 1987, Pages 474-478. The isolated polynucleotide encoding the wild type polypeptide may be cloned into a vector, such as the pET28a plasmid.

An alternative method to producing a polynucleotide with a desired sequence is the use of a synthetic gene. A polynucleotide encoding a polypeptide of the present invention may be prepared synthetically, for example using the phosphoroamidite method (see, Beaucage et al., Curr Protoc Nucleic Acid Chem. 2001 May; Chapter 3:Unit 3.3; Caruthers et al., Methods Enzymol. 1987, 154:287-313).

The polynucleotide thus produced may then be subjected to further manipulations, including one or more of purification, annealing, ligation, amplification, digestion by restriction endonucleases and cloning into appropriate vectors. The polynucleotide may be ligated either initially into a cloning vector, or directly into an expression vector that is appropriate for its expression in a particular host cell type.

The polynucleotides may include non-coding sequences, including for example, non-coding 5′ and 3′ sequences, such as transcribed, non-translated sequences, termination signals, ribosome binding sites, sequences that stabilize mRNA, introns and polyadenylation signals. The polynucleotides may comprise coding sequences for additional amino acids heterologous to the variant polypeptide, in particular a marker sequence, such as a poly-His tag, that facilitates purification of the polypeptide in the form of a fusion protein.

Polypeptides may be produced as tagged proteins, for example to aid in extraction and purification. A non-limiting example of a tag construct is His-Tag (six consecutive histidine residues), which can be isolated and purified by conventional methods. It may also be convenient to include a proteolytic cleavage site between the tag portion and the protein sequence of interest to allow removal of tags, such as a thrombin cleavage site.

The polynucleotide encoding the polypeptide may be incorporated into a wide variety of expression vectors, which may be transformed into in a wide variety of host cells. The host cell may be prokaryotic or eukaryotic.

Introduction of a polynucleotide into the host cell can be effected by well known methods, such as chemical transformation (e.g. calcium chloride treatment), electroporation, conjugation, transduction, calcium phosphate transfection, DEAE-dextran mediated transfection, transvection, microinjection, cationic lipid-mediated transfection, scrape loading, ballistic introduction and infection.

In some embodiments, the cell is a prokaryotic cell. Representative, non-limiting examples of appropriate prokaryotic hosts include bacterial cells, such as cells of Escherictahia coli and Bacillus subtilis. In other embodiments, the cell is a eukaryotic cell. In some exemplary embodiments, the cell is a fungal cell, such as yeast. Representative, non-limiting examples of appropriate yeast cells include Saccharomyces cerevisiae and Pichia pastoris. In additional exemplary embodiments, the cell is a plant cell.

The polypeptides may be expressed in any vector suitable for expression. The appropriate vector is determined according the selected host cell. Vectors for expressing proteins in E. coli, for example, include, but are not limited to, pET, pK233, pT7 and lambda pSKF. Other expression vector systems are based on beta-galactosidase (pEX); maltose binding protein (pMAL); and glutathione S-transferase (pGST).

Selection of a host cell transformed with the desired vector may be accomplished using standard selection protocols involving growth in a selection medium which is toxic to non-transformed cells. For example, E. coli may be grown in a medium containing an antibiotic selection agent; cells transformed with the expression vector which further provides an antibiotic resistance gene, will grow in the selection medium.

Upon transformation of a suitable host cell, and propagation under conditions appropriate for protein expression, the desired polypeptide may be identified in cell extracts of the transformed cells. Transformed hosts expressing the polypeptide of interest may be identified by analyzing the proteins expressed by the host using SDS-PAGE and comparing the gel to an SDS-PAGE gel obtained from the host which was transformed with the same vector but not containing a nucleic acid sequence encoding the protein of interest.

The protein of interest can also be identified by other known methods such as immunoblot analysis using suitable antibodies, dot blotting of total cell extracts, limited proteolysis, mass spectrometry analysis, and combinations thereof.

The protein of interest may be isolated and purified by conventional methods, including ammonium sulfate or ethanol precipitation, acid extraction, salt fractionation, ion exchange chromatography, hydrophobic interaction chromatography, gel permeation chromatography, affinity chromatography, and combinations thereof.

The isolated protein of interest may be analyzed for its various properties, for example specific activity and thermal stability, using methods known in the art, some of them are described hereinbelow.

Conditions for carrying out the aforementioned procedures as well as other useful methods are readily determined by those of ordinary skill in the art (see for example, Current Protocols in Protein Science, 1995 John Wiley & Sons).

In particular embodiments, the polypeptides of the invention can be produced and/or used without their start codon (methionine or valine) and/or without their leader (signal) peptide to favor production and purification of recombinant polypeptides. It is known that cloning genes without sequences encoding leader peptides will restrict the polypeptides to the cytoplasm of the host cell and will facilitate their recovery (see for example, Glick, B. R. and Pasternak, J. J. (1998) In “Molecular biotechnology: Principles and applications of recombinant DNA”, 2nd edition, ASM Press, Washington D.C., p. 109-143).

The present invention further provides compositions comprising the multi-enzyme complex of the present invention, for use in biomass degradation.

The present invention further provides genetically-modified cells capable of producing the multi-enzyme complex of the present invention. These cells are capable of producing, and typically secreting, the different components of the complex.

In some embodiments, the genetically-modified cell is selected from a prokaryotic and eukaryotic cell. Each possibility represents a separate embodiment of the invention.

The present invention provides systems for bioconversion of cellulosic material, the system comprising the multi-enzyme complex of the present invention.

Methods and Uses

The multi-enzyme complexes of the present invention, compositions comprising same and cells producing same may be utilized for the bioconversion of a cellulosic material into degradation products.

“Cellulosic materials” and “cellulosic biomass” refer to materials that contain cellulose, in particular materials derived from plant sources that contain cellulose. The cellulosic material encompasses ligno-cellulosic material containing cellulose, hemicellulose and lignin. The cellulosic material may include natural plant biomass and also paper waste and the like. Examples of suitable cellulosic materials include, but are not limited to, wheat straw, switchgrass, corn cob, corn stover, sorghum straw, cotton straw, bagasse, energy cane, hard wood paper, soft wood paper, or combinations thereof.

Resulting sugars may be used for the production of alcohols such as ethanol, propanol, butanol and/or methanol, production of fuels, e.g., biofuels such as synthetic liquids or gases, such as syngas, and the production of other fermentation products, e.g. succinic acid, lactic acid, or acetic acid.

According to an aspect of the present invention, there is provided herein a method for converting cellulosic material into degradation products, the method comprising exposing said cellulosic material to the multi-enzyme complex of the present invention.

In some embodiments, assembling the multi-enzyme complex prior to contacting with the cellulosic material comprises the following steps: (i) mixing in a first solution a first scaffold polypeptide with its corresponding enzymes to obtain a first scaffold-enzyme complex; (ii) mixing in a second solution a second polypeptide with its corresponding enzymes to form a second scaffold-enzyme complex; and (iii) mixing the first and second solution to obtain binding of the first and second scaffolds, to thereby obtain a multi-enzyme complex of the present invention.

According to an additional aspect of the present invention, there is provided herein a method for converting cellulosic material into degradation products, the method comprising exposing said cellulosic material to genetically-modified cells capable of producing the multi-enzyme complex of the present invention.

The degradation products typically comprise mono-, di- and oligosaccharide, including but not limited to glucose, xylose, cellobiose, xylobiose, cellotriose, cellotetraose, arabinose, xylotriose.

Multi-enzyme complexes of the present invention may be added to bioconversion and other industrial processes, for example, continuously, in batches or by fed-batch methods. Alternatively or additionally, the multi-enzyme complexes of the invention may be recycled. By relieving end-product inhibition of endoxylanases and exo/endoglucanases (such as xylobiose and cellobiose), it may be possible to further enhance the hydrolysis of the cellulosic material.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Effect of Linker Length in a Scaffold Polypeptide

Preparation of a Combinatorial Library of Scaffold Polypeptides:

A combinatorial library of recombinant trivalent designer scaffold polypeptides was prepared. The scaffold library was prepared from the following four modules:
a carbohydrate binding module (designated “CBM”): CBM3a of CipA from C. thermocellum (GenBank Accession No. ABN54273) (SEQ ID NO: 1)
three (3) divergent cohesin modules of different specificities:

(i) cohesin from C. thermocellum (the second cohesin of CipA from C. thermocellum, designated “Ct” or “T”) (GenBank Accession No. ABN54273) (SEQ ID NO: 2)

(ii) cohesin from B. cellulosolvens (the third cohesin of ScaB from B. cellulosolvens, designated “Bc” or “B”) (GenBank Accession No. AAT79550) (SEQ ID NO: 3)

(iii) cohesin from A. cellulolyticus (the third cohesin of ScaC from A. cellulolyticus, designated “Ac” or “A”) (GenBank Accession No. AAP48996). (SEQ ID NO: 4)

The library was designed such that the different modules are separated by linkers of 0 (“no linker”), 5 (“short”) or 27-35 (“long”) amino acids. The amino-acid content of the different linkers used in this work is shown in Table 1

TABLE 1
Set of inter-modular
linkers used for cloning

| | | | C- | | | |
|---|---|---|---|---|---|---|
| Source | | | term. | Length | Sequence | Accession |
| organism | Scaf. | Module | linker | (a.a) | (SEQ ID NO.) | code |
| A. | Sca | Coh A | Long | 29 | PTPTQSATPTVT | AAP48996 |
| cellulolyticus | C | | | | PSATATPTQSAT | |
| | | | | | PTVTP (5) | |
| | | | Short | 5 | PTPTQ (6) | |
| | | | No | — | — | |
| B. | Sca | Coh B | Long | 27 | TPTNTISVTPTN | AAT79550 |
| cellulosolvens | B | | | | NSTPTNNSTPKP | |
| | | | | | NPL (7) | |
| | | | Short | 5 | TPTNT (8) | |
| | | | No | — | — | |
| C. | Cip | Coh T | Long | 35 | PTKGATPTNTAT | ABN54273 |
| thermocellum | A | | | | PTKSATATPTRP | |
| | | | | | SVPTNTPTNTP | |
| | | | | | (9) | |
| | | | Short | 5 | PTKGA (10) | |
| | | | No | — | — | |
| | | CBM | Long | 31 | VVPSTQPVTTPP | ABN54273 |
| | | | | | ATTKPPATTKPP | |
| | | | | | ATTIPPS (11) | |
| | | | Short | 5 | VVPST (12) | |
| | | | No | — | — | |

The preceding module of each linker is indicated.

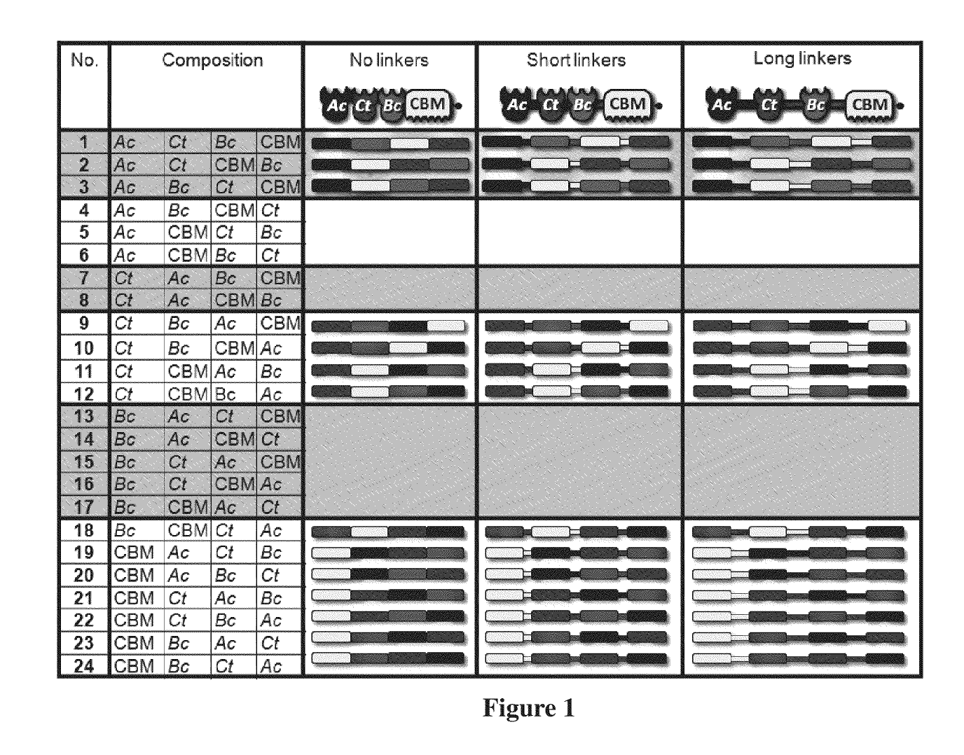

In principle, the four modules could be shuffled to result in 24 different arrangements, each with linkers of three different lengths separating the modules. Therefore, from the basic scaffold template, 72 possible combinations could potentially be produced. Fourteen (14) full sets, representing 42 cloned and expressed scaffoldins, were successfully cloned and expressed and used for further study. FIG. 1 specifies the 72 possible combinations. Only complete sets are shown in a modular schematic representation.

Details about the cloning, expression and purification of the different scaffold polypeptides in the library are given below (“Material and methods”).

Assembly of Designer Cellulosomes:

To assemble designer cellulosomes, the following three model cellulases from C. thermocellum were used: the exoglucanase Cel48S together with its native dockerin (designated as “48S-t”), the endoglucanase Cel8A fused to a dockerin module of ScaA from B. cellulosolvens (designated “8A-b”), and the proccessive-endoglucanase Cel9K fused to a dockerin module of ScaB from A. cellulolyticus (designated as “9K-a”).

The construction of the recombinant enzymes is described below.

The amino acid sequence of 48S-t is set forth in SEQ ID NO: 13. The dockerin module corresponds to residues 652-715 of the sequence. The polynucleotide sequence encoding 48S-t is set forth in SEQ ID NO: 14.

The amino acid sequence of 8A-b is set forth in SEQ ID NO: 15. The dockerin module corresponds to residues 389-459 of the sequence. The polynucleotide sequence encoding 8A-b is set forth in SEQ ID NO: 16.

The amino acid sequence of 9K-a is set forth in SEQ ID NO: 17. The dockerin module corresponds to residues 808-878 of the sequence. The polynucleotide sequence encoding 9K-a is set forth in SEQ ID NO: 18.

In summary, the following multi-enzyme configuration was tested:
Scaffold composed of:
Cohesin modules from C. thermocellum, B. cellulosolvens, and A. cellulolyticus; CBM;

where the different modules are separated by linkers of 0 (no linker), 5 or 27-35 amino acids.
Enzymes:

Cel48S (C. thermocellum)+dock from C. thermocellum (designated as “4854”)

Cel8A (C. thermocellum)+dock from B. cellulosolvens (designated as “8A-b”)

Cel9K (C. thermocellum)+dock from A. cellulolyticus (designated as “9K-a”)

The specificity of the cohesin-dockerin interaction was verified by affinity-based ELISA as will be detailed below. The chimaeric scaffolds were found to interact specifically with their matching dockerins. Likewise, the cellulases interacted specifically with their matching cohesin.

The formation of designer-cellulosome complexes was initially analyzed by non-denaturing PAGE. Molar ratios for complete interaction of each enzyme were determined with several representative scaffolds from the scaffold set. These predetermined molar ratios were used for the interaction of the three enzymes with the entire 42 scaffoldin set, and non-denaturing PAGE was used to evaluate the resultant complexes. Each complex migrated on the gel as a major band, shifted from the bands of the individual components of the designer cellulosome, indicating a productive near-complete or complete interaction in each case. In addition, the designer cellulosome complexes were analyzed by size exclusion chromatography, whereby each of the single components was assessed separately, and their retention volume was used as marker for analysis of the designer cellulosome complexes. Cellulosome complexes eluted faster than the single enzymes and scaffolds, appearing as a major peak. Fractions from the designer cellulosome complexes were pooled, concentrated and then analyzed by SDS-PAGE. The major peak was shown to consist of all three enzymes together with the chimaeric scaffold.

Activity Assays:

In a preliminary assay, the recombinant enzymes were tested for their ability to degrade phosphoric-acid swollen cellulose (PASC) or Avicel, and their activities were comparable to those of the wild-type enzymes.

The activities of designer cellulosomes were examined using Avicel as a pure microcrystalline cellulose substrate and pretreated cellulose-enriched wheat straw, containing 90% cellulose, 5% hemicellulose and 5% lignin, as a model substrate derived from a native source.

A preliminary kinetics assay with one representative scaffold set was performed in order to determine the end-point for the cellulose hydrolysis reaction on either substrate.

Next, cellulose hydrolysis by designer cellulosomes composed of each of the scaffolds in the library was tested, at a single time point (pre-determined by the kinetics assay). For Avicel, activity was tested at 72 hours, since shorter incubation times had lower than 5% conversion rates. For pretreated wheat straw the kinetics reaction reached a conversion of about 20% after 3 hours, thus longer incubation times were unnecessary.

Activity was tested for the following combinations:
a. A mixture of the three enzymes in a free state
b. A mixture of the three enzymes in a free state, where each enzyme is bound to a mini-scaffold composed of a matching cohesin module and a CBM (“CBM-Coh”). Thus, the enzymes are not integrated into one complex, but each enzyme is targeted separately to the substrate
c. A complex of the three enzymes bound to a scaffold from the library. Fourteen different scaffold arrangements (sets) were tested on Avicel and pretreated wheat straw for their activities in combination with the three cellulases; for each arrangement three scaffolds were tested that vary in the length of the intermodular linkers, namely no linkers, 5 amino acids or an average of 30.5 (27-35) amino acids.

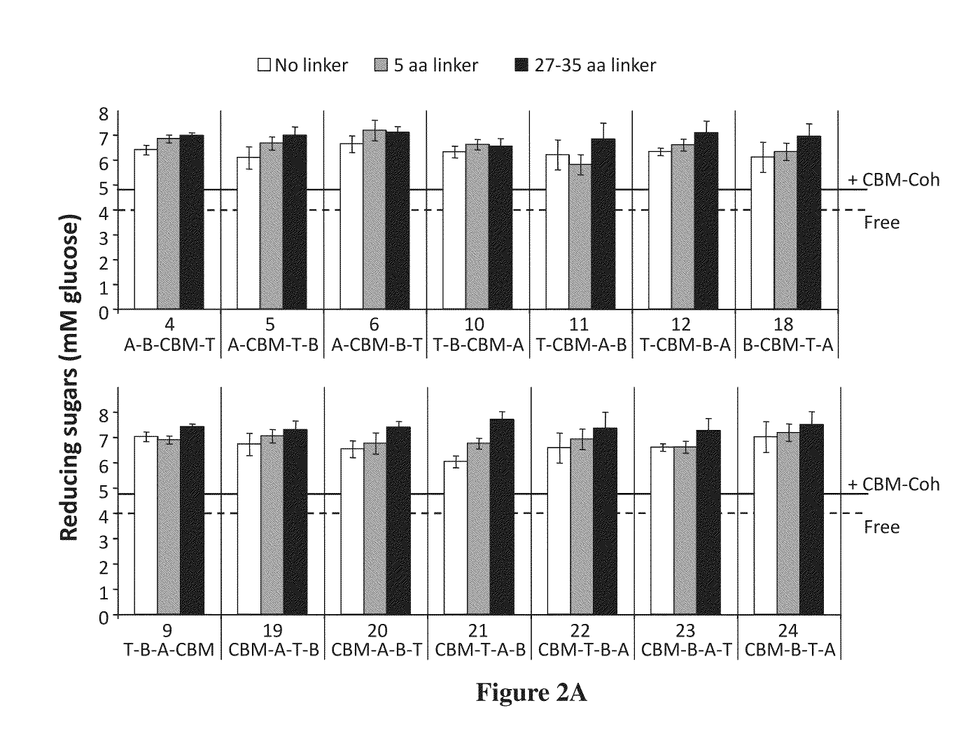

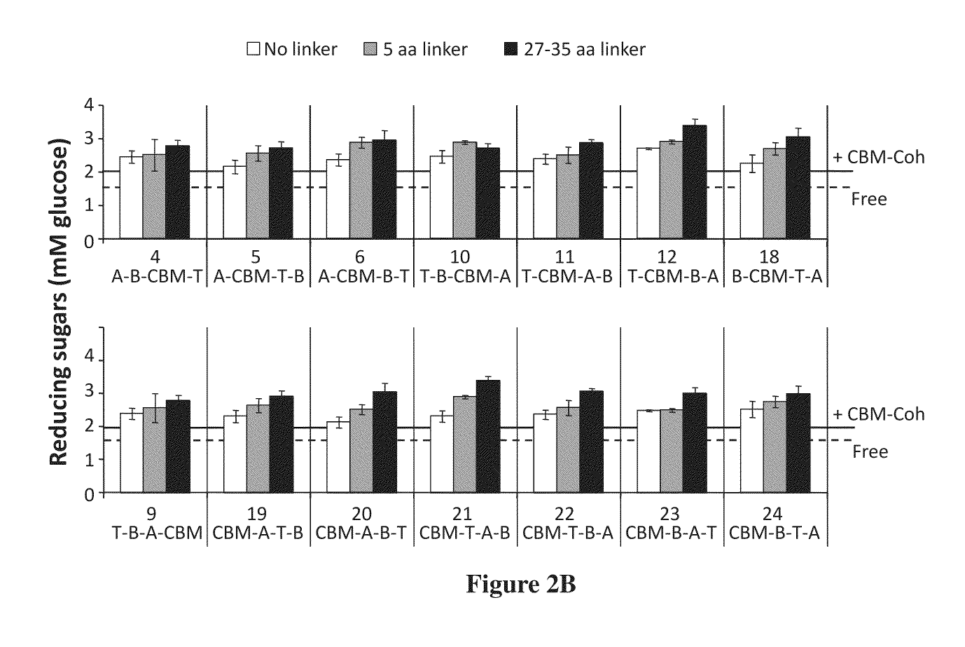

The results are shown in FIG. 2A (Avicel) and FIG. 2B (pretreated wheat straw). In both A and B, the upper panels show the activities of the cellulosomes having scaffold sets with internal CBMs, and the lower panels provide the results of cellulosomes with scaffolds bearing CBMs at the extremities.

All combinations of modular arrangements and intermodular linker lengths of the designer scaffold yielded active trivalent designer cellulosome assemblies on both substrates. The designer cellulosomes showed a synergistic effect and had a higher activity compared to the free enzymes as well as the targeted enzymes (via CBM-Cohs).

The results also revealed a trend of increased activity on both substrates, as the intermodular linker length increased from no linkers at all to short 5-residue linkers, and from short to long linkers. Two-way ANOVA with interaction was used for statistical verification with length and the 14 scaffoldin arrangements as factors; no interaction was found for either substrate [Avicel (p=0.16), pretreated wheat straw (p=0.0595)], indicating that linker length indeed had a significant effect on activity. The activities exhibited by the long, short and no-linker scaffolds were thus observed to be significantly different from each other in the majority of the sets for both substrates.

A preferred modular arrangement for the trivalent designer scaffold was not observed for the three enzymes used in this study, indicating that these three enzymes could be integrated at any position in the designer cellulosome without significant effect on cellulose-degrading activity.

Materials and Methods

1. Cloning of Cellulases

The recombinant wild-type family-48 exocellulase, Cel48S-ct, was amplified from C. thermocellum ATCC 27405 genomic DNA with the following forward and reverse primers: 5′ CAGTCCATGGGTCCTACAAAGGCACCTAC 3′ (SEQ ID NO: 19) and 5′ CGCGAAGCTTTTAATGGTGATGGTGATGGTGG 3′ (SEQ ID NO: 20), respectively (NcoI and HindIII restriction sites in bold), that allow incorporation into pET28a. Similarly, the recombinant wild-type family-8 endocellulase, Cel8A-bc Cel8A was cloned from the genomic DNA of C. thermocellum with the following forward and reverse primers, 5′ CAGTCCATGGGTGTGCCTTTTAACACAAA 3′ (SEQ ID NO: 21) and 5′ CACGCTCGAGATAAGGTAGGTGGGGTATGC 3′ (SEQ ID NO: 22) respectively, (NcoI and XhoI restriction sites in bold). Likewise, the recombinant wild-type family-9 endocellulase, Cel9K-ct, was amplified from the C. thermocellum genomic DNA and cloned into pET28a vector using the restriction free (RF) method (Unger et al., 2010, J Struct Biol. 172:34-44) with the following forward and reverse primers,

(SEQ ID NO: 23)
5′ GTTTAACTTTAAGAAGGAGATATACCATGGGCCATCACCATCAC
CATCACTTAGAAGACAAGTCTCCAAAGTTGCCGGAT 3′
and
(SEQ ID NO: 24)
5′ GAGTGCGGCCGCAAGCTTGTCGACGGAGCTCTTATTTATGTGGC
AATACATCTATCTCTTTAAG 3′

respectively, (gene specific sequences are underlined, plasmid specific sequences are shown in plain font, His-tag in bold). For the cloning of Cel9K-ac with the divergent dockerin from A. cellulolyticus, the dockerin was amplified from the genomic DNA of A. cellulolyticus and used for the simultaneous insertion of the divergent dockerin and deletion of the wild-type dockerin into the wild-type Cel9K-ct plasmid using the RF cloning method with the following forward and reverse primers,

(SEQ ID NO: 25)
5′ CTCGATGAAATTGACTTAATAACACCGCCAGGTACCAAATTTAT
ATATGGTGATGTTGATGGTAATG 3′
and
(SEQ ID NO: 26)
5′ GAGTGCGGCCGCAAGCTTGTCGACGGAGCTCTTATTCTTCTTTC
TCTTCAACAGGGAATAAAAATATC 3′

respectively (gene specific sequences are underlined, plasmid specific sequences are in regular case). For the cloning of the chimaeric enzyme Cel8A-bc with the C. thermocellum Cel8A catalytic module and a divergent dockerin from B. cellulosolvens, the catalytic module of Cel8A was amplified from C. thermocellum ATCC 27405 genomic DNA with the following forward and reverse primers, 5′ ATTCAACCATGGGTGTGCCTTTTAACACAAAATAC 3′ (SEQ ID NO: 27) and 5′ ATATTGCTCGAGTAATGTGGTACCAATGAAGGTGTCGGATTCGACG 3′ (SEQ ID NO: 28) respectively (NcoI, KpnI and XhoI restriction sites in bold case). The PCR product was cloned into a pET28a plasmid linearized with NcoI and XhoI restriction enzymes to yield p8A-CD. The dockerin was amplified from B. cellulosolvens genomic DNA with the following forward and reverse primers, 5′ ACTTTAGGTACCTCCAAAAGGCACAGCTAC 3′ (SEQ ID NO: 29) and 5′ ATTAATCTCGAGCGCTTTTTGTTCTGCTGG 3′ (SEQ ID NO: 30) respectively (KpnI and XhoI restriction sites in bold case). The resultant DNA was cloned into p8A-CD that was linearized with KpnI and XhoI to yield p8A-bc.

2. High-Throughput Computer-Aided Cloning of Short- and No-Linker Scaffolds

A computer-aided, automated method for combinatorial DNA library design and production was employed for the construction and cloning of the scaffolds which either lacked intermodular linkers or contained short (5 aa) intermodular linkers. The design and synthesis of the scaffolds using this approach were performed using computer-aided methods for specifying, visualizing and planning and executing the actual production of the desired DNA libraries (Linshiz et al., 2008, Mol Syst Biol, 4:191; and Shabi et al., 2010, Syst Synth Biol, 4:227-236).

The core recursive construction step in this method required four basic enzymatic reactions: phosphorylation, elongation, PCR and Lambda exonucleation, and was performed as previously described by Linshiz noted above using a set of primers designed for this purpose.

The PCR product was amplified in order to yield sufficient amounts of DNA for subsequent cloning, by a second set of primers, according to the modules that were located at the 5′ and 3′ of each scaffold construct. The amplified product was digested by NcoI and XhoI, and ligated with NcoI-XhoI linearized pET28a vector (Novagene, Madison, Wis.). Positive clones were selected by colony PCR and verified by sequencing.

3. Restriction-Free (RF) Cloning of Long-Linker Scaffolds

A second approach, involving restriction-free multi-component assembly of DNA segments (Unger et al., 2010, J Struct Biol, 172:34-44), was used for cloning the scaffolds with long (27-35 aa) intermodular linkers. For the construction of each scaffold, 8 primer pairs were designed. A His-tag was added at the C terminus of each construct for further purification using a Ni-nitrilotriacetic acid (NTA) column (Qiagen GmbH, Hiden, Germany). The four modules were amplified by PCR with 25- to 30-bp overhangs on both the 5′ and 3′ ends, corresponding to the adjoining regions (either with another adjoining insert gene or with the expression vector as needed). Next, the PCR products served as mega-primers for simultaneous assembly of the vector (pET28a plasmid) and inserts by linear amplification, resulting in a linear plasmid (pET28a) containing a sequence encoding a recombinant scaffold polypeptide with four modules.

Primer sets were designed for PCR amplification and subsequent RF reactions were carried out using Phusion polymerase (Thermo Scientific).

4. Expression and Purification of Cellulases and Designer Scaffolds

Escherichia coli BL21 (DE3) cells overproducing pET28a-scaffold genes or cellulases were grown at 37° C. in Luria-Bertani broth supplemented with 50 μg/ml kanamycin (Sigma-Aldrich Chemical Co, St. Louis, Mo.) to A₆₀₀=0.8-1.0. The cultures were cooled to 16° C., and protein expression was induced by the addition of 0-1 mM isopropyl-1-thio-β-D-galactoside—IPTG (Fermentas UAB Vilnius, Lithuania), based on the results of predetermined optimization experiments. The cultures were incubated at 16° C. for additional 16 h, the cells were harvested by centrifugation (3500 g, 15 min), resuspended in Tris-buffered saline (TBS, 137 mM NaCl, 2.7 mM KCl, 25 mM Tris-HCl, pH 7.4) supplemented with 5 mM imidazole (Merck KGaA, Darmstadt, Germany) and disrupted by sonication. The sonicate was heated for 20 min to 60° C. and centrifuged (20,000 g, 30 min). The supernatant fluids were mixed with 4 ml of Ni-NTA beads for 1 h on a 20-ml Econo-pack column for batch purification at 4° C. The column was washed by gravity flow with 100 ml wash buffer (TBS, 50 mM imidazole) and elution was performed with 14 ml of elution buffer (TBS, 250 mM imidazole). For purification of the scaffolds an additional affinity-purification step was applied: the eluted fractions were incubated in a 50-ml tube with 10 ml phosphoric-acid swollen cellulose (PASC) (0.75 mg/ml) for 1 h at 4° C. to allow binding of the CBM. The matrix was washed three times with TBS, containing 1 M NaCl, and three times with TBS without added salt. The scaffold was eluted with 1% triethylamine and neutralized with 1 M 2-(N-Morpholino)ethanesulfonic acid (MES) buffer pH 5. For both scaffolds and cellulases the buffer was exchanged by dialysis against TBS, and the scaffold sample was concentrated using Amicon Ultra 15 ml 50,000 MWCO concentrators (Millipore, Bedford, Mass.). Protein concentrations were estimated by the absorbance at 280 nm. Extinction coefficient was determined based on the known amino acid composition of each protein using the ProtParam tool on the EXPASY server (http://www.expasy.org/tools/protparam.html) (Gasteiger et al., 2005, Protein Identification and Analysis Tools on the ExPASy Server).

5. Analysis of Cohesin-Dockerin Specificity

The procedure of Barak et al, 2005, J Mol Recogit, 18:491-501 was followed with minor modifications. Maxisorp ELISA plates (Nunc A/S, Roskilde, Denmark) were coated with 1 μg/ml each of the dockerin-containing enzymes Cel48S-ct, Cel9K-ac and Cel8A-bc, and then interacted with 0.1-1000 ng/μl of its matching CBM-cohesin (CBM-CohCt A2, CBM-CohAc C3 and CBM-Coh-Bc B3) counterpart. Rabbit-anti-CBM (diluted 1:3000 in blocking buffer) was used as primary antibody for detection of the interaction. For analysis of the chimaeric scaffolds, Maxisorp ELISA plates were coated with 1 μg/ml of the chimaeric scaffold and then interacted with 0.1-1000 ng/μl of matching Xyn-Doc proteins which were prepared as described in Barak et al, 2005 noted above. These proteins are composed of xylanase T-6 from Geobacillus stearothermophilus fused to a dockerin module of appropriate specificity. Rabbit anti-xylanase T-6 antibody diluted 1:10,000 in blocking buffer) was used as primary antibody for detection of the interaction. A Secondary Antibody Preparation of Goat-HRP-labeled anti-rabbit antibody diluted 1:10,000 was added. The interaction was detected using TMB Substrate-Chromogen (Dako A/S, Glostrup, DK), and the reaction was terminated by the addition of 1 M H₂SO₄. Absorbance was measured at 450 nm

6. Non-Denaturing PAGE

Equimolar concentrations of scaffolds and matching enzymes (4-8 μg each protein) were mixed and added to similar volumes of interaction buffer (TBS with 10 mM CaCl₂ and 0.05% Tween20). DDW was added to a final volume of 30 μl. The proteins were incubated at 37° C. for 2 h to allow complex formation. Non-denaturing sample buffer (192 mM glycine, 25 mM Tris) was added, and a total of 15 μl/lane was subjected to PAGE (7.5-9% acrylamide gels), using a Bio-Rad power pack 300. Single components (scaffold and enzymes) were used as markers. The remaining 15 μl were used for analysis on SDS-PAGE

7. Size-Exclusion High Performance Liquid Chromatography (HPLC)

Equimolar protein concentrations (450 picomoles scaffold or enzyme) were diluted in 300 μl of loading buffer (Tris Buffered Saline pH=7.4 (TBS), supplemented with 2 mM of CaCl₂). For the formation of designer cellulosome complexes, equimolar concentrations of a scaffold and enzymes were incubated at 37° C. for 2 h with similar volumes of interaction buffer (TBS with 10 mM CaCl₂ and 0.05% Tween20), and loading buffer was added to a final volume of 300 μl. The reactions were injected onto an analytical Superdex 200 HR 10/30 column using an AKTA fast-performance liquid chromatography system (GE Healthcare, Uppsula, Sweden) and loading buffer at a flow rate of 0.5 ml·min⁻¹. Eluted proteins were detected at 280 nm and fractions (0.5 ml) concentrated and analyzed using SDS-PAGE gels.

8. Preparation of Cellulose-Enriched (Pretreated) Wheat Straw

Wheat straw was cut into pieces and ground to obtain a powder with an average particle size of 1-3 mm. A sample (20 g) of the resultant powder was treated with 85 ml of 5% (v/v) nitric acid for 1 h at 115° C. The acid-treated biomass was washed with DDW and treated further with 150 ml of 1.5% v/v NaOH for 1 h at 100° C. and washed with DDW, yielding a cellulose-enriched substrate.

9. Determination of Wheat Straw Substrate Chemical Composition

The chemical composition of the samples was determined according to the following improvement of the TAPPI-method. For hemicellulose content, samples were boiled with 2% HCl for 2 h, washed with DDW and ethanol and dried at 105° C. to constant weight (about 2-3 h). For cellulose content, samples were boiled with an ethanolic HNO₃ solution for 1 h, washed with DDW and ethanol, and dried at 105° C. to constant weight (about 2-3 h). For lignin content, samples were swollen in 72% H₂SO₄ at room temperature for 2 h, diluted with DDW to 8-10% acid, hydrolyzed with boiling diluted H₂SO₄ (8-10%) for 2 h, washed with DDW and ethanol, and dried at 105° C. to constant weight (about 2-3 h). Total solid content was determined by drying the samples at 105° C. for 2 h.

10. Activity Assays

The hydrolysis reactions were carried out in a total volume of 200 μl, and consisted of reaction buffer (100 mM sodium acetate buffer pH 5.5, 24 mM CaCl₂, 4 mM EDTA), 0.5 μM of each protein and 2% w/v Avicel (Sigma-Aldrich Chemical Co, St. Louis, Mo.) or 3.5 gr/L pretreated (cellulose-enriched) wheat straw. Prior to the addition of the substrate, each scaffold was incubated with equimolar quantities of the three enzymes for 2 h at 37° C. with a similar volume of interaction buffer (TBS with 10 mM CaCl₂ and 0.05% Tween 20). The reaction was carried out for 24-72 h (Avicel) or 3-24 hours (pretreated wheat straw) at 50° C. and terminated by immersion in ice water. The substrate was pelleted by centrifugation at maximum speed (20,800×g, 10-15 min), and 100 μl of the supernatant was transferred to a new tube. Dinitrosalycylic acid (DNS, 150 μl) was added, and the samples were boiled for 10 min. The absorbance was measured at 540 nm and the reducing sugars were determined according to a glucose calibration curve. Each assay was repeated three times in triplicate.

### Example 2

### Construction and Testing of an Artificial Cellulosome Complex Composed of Primary and Adaptor Scaffold Polypeptides

Construction of an Adaptor Scaffold

An adaptor scaffold was prepared which includes the following modules separated by linkers of 27-35 amino acids: three divergent cohesin modules from A. cellulolyticus (the third cohesin of ScaC noted above, designated “A”), B. cellulosolvens (the third cohesin of ScaB noted above, designated “B”) and C. thermocellum (the second cohesin of CipA noted above, designated “T”) for integration of enzymes, a type II dockerin module from C. thermocelum (from CipA, UniProtKB/Swiss-Prot Accession No. Q46453, designated “DockII”) for attachment to a primary scaffold, and a CBM from C. thermocellum (CBM3a of CipA noted above, designated “CBM”). The amino acid sequence of the adaptor scaffold is set forth in SEQ ID NO: 31. The polynucleotide sequence encoding the adaptor scaffold is set forth in SEQ ID NO: 32.

The adaptor scaffold was designed to interact with the following three enzymes:
Cel9A (T. fusca) (processive endoglucanase) with a dockerin from A. cellulolyticus (dockerin of ScaA) (designated as “a-9A”)
Cel48A (T. fusca) (exoglucanase) with a dockerin from B. cellulosolvens (designated as “b-48A”)
Cel5A (T. fusca) (endoglucanase) with a dockerin from C. thermocellum (dockerin of C. thermocellum xylanase Xyn10Z) (designated as “5A-t”).

The construction of the recombinant Cel48A and Cel5A is described in Caspi et al., 2008, Journal of Biotechnology, 135: p. 351-357; and Caspi et al., 2009, Applied and Environmental Microbiology, 75: p. 7335-7342. The recombinant Cel9A was constructed by removing CBM2 of the wild type enzyme at the C-terminus and adding a dockerin module from A. cellulolyticus (from ScaB) at the N-terminus. A His-tag was added at the beginning of the sequence. The protein was purified using conventional Nickel beads purification protocol.

The amino acid sequence of a-9A is set forth in SEQ ID NO: 33. The dockerin module corresponds to residues 16-86 of the sequence. The polynucleotide sequence encoding a-9A is set forth in SEQ ID NO: 34.

The amino acid sequence of b-48A is set forth in SEQ ID NO: 35. The dockerin module corresponds to residues 18-88 of the sequence. The polynucleotide sequence encoding b-48A is set forth in SEQ ID NO: 36.

The amino acid sequence of 5A-t is set forth in SEQ ID NO: 37. The dockerin module corresponds to residues 313-376 of the sequence. The polynucleotide sequence encoding 5A-t is set forth in SEQ ID NO: 38.

Preliminary experiments with nine (9) scaffold polypeptides from the library described in Example 1, which include different arrangements of the selected cohesin and CBM modules noted above, were performed in order to determine the modular arrangement which provides better overall cellulolytic activity. The nine scaffolds that were examined are shown in Table 2. Each scaffold was interacted with the three enzymes noted above and the activity on Avicel was tested. An additional experiment was performed with an adaptor scaffold which includes a dockerin type II from C. thermocellum and cohesins type I from A. cellulolyticus, B. cellulosolvens and C. thermocellum but lacks a CBM (designated “DockII-A-B-T”). This scaffold was targeted to the substrate via interaction with a mini-scaffold containing a CBM fused to a type II cohesin matching the type II dockerin of the adaptor scaffold.

Activity of the different adaptor-enzyme complexes was compared to that of a mixture of free enzymes and a mixture of the enzymes where each enzyme is bound to a cohesin-CBM mini-scaffold (matching the enzyme-borne dockerin).

TABLE 2

| No. | Composition |
|---|---|
| 4 (long) | A-B-CBM-T |
| 5 (long) | A-CBM-T-B |
| 9 (long) | T-B-A-CBM |
| 18 (long) | B-CBM-T-A |
| 19 (long) | CBM-A-T-B |
| 20 (long) | CBM-A-B-T |
| 21 (long) | CBM-T-A-B |
| 22 (long) | CBM-T-B-A |
| 23 (long) | CBM-B-A-T |

The adaptor scaffold with the sequence set forth in SEQ ID NO: 31 was selected for further study following the preliminary experiments. In this adaptor scaffold the modules are arranged as follows: CBM-cohesins A-B-T-DockII (designated “CBM-A-B-T-DockII”). This adaptor integrates the three enzymes such that Cel9A is adjacent to the CBM positioned at one terminus of the scaffold, Cel48A is in the middle, and Cel5A is positioned at the other terminus of the scaffold, adjacent to the type II dockerin.

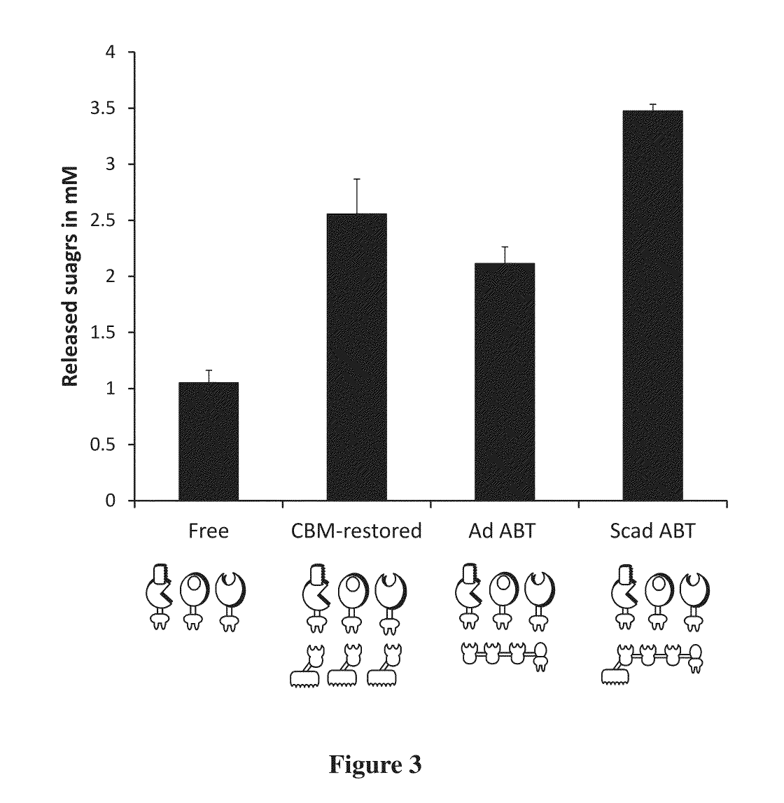

Activity assays on Avicel showed targeting and proximity effects resulting in improved cellulolytic activity compared to a mixture of free enzymes, mixture of enzymes bound to matching cohesin-CBM mini-scaffolds, and the enzymes bound to the an adaptor DockII-A-B-T (lacking a CBM), which is further bound to a matching cohesin-CBM mini-scaffold (FIG. 3).

Multi Enzyme Complex Containing Primary and Adaptor Scaffolds

A primary scaffold was prepared, which is able to interact with the adaptor scaffold CBM-A-B-T-DockII described above. The primary scaffold was prepared as a hexavalent scaffold containing six cohesin modules that can integrate six dockerin-bearing subunits. In particular, a hexavalent scaffold was prepared for integration of five (5) carbohydrate active enzymes and one adaptor scaffold. Altogether, a complex of the adaptor and primary scaffolds can integrate eight (8) carbohydrate active enzymes (five on the primary scaffold and three on the adaptor scaffold).

Primary Hexavalent Scaffold:
Scaffold:

The scaffold was prepared from the following modules:
Cohesin from C. cellulolyticum (cohesin 1 from scaffoldin CipC, GenBank Accession No. U40345.3) (designated “C”) (SEQ ID NO: 39);
Cohesin from, A. cellulolyticus (cohesin 3 from scaffoldin C noted above) (designated “A”);
Cohesin from C. thermocellum (cohesin 3 from the cellulosomal scaffoldin subunit CipA noted above) (designated “T”);
Cohesin from Archaeoglobus fulgidus (cohesin 2375, GenBank Accession No. AE001112.1) (designated “G”) (SEQ ID NO: 40);
Cohesin from Ruminococcus flavefaciens (cohesin 1 from scaffoldin B of strain 17, GenBank Accession No. AJ278969.4) (designated “F”) (SEQ ID NO: 41);
Cohesin type II from C. thermocellum from OlpB (NCBI Reference Sequence: YP_001039467 YP001039467 or UniProtKB Accession Number Q06852) (designated “CohII”) (SEQ ID NO: 42);
CBM (C. thermocellum, CBM3a of CipA noted above) (designated “CBM”).

The amino acid sequence of the primary scaffold is set forth in SEQ ID NO: 43. The polynucleotide sequence encoding the primary scaffold is set forth in SEQ ID NO: 44. In this primary scaffold the modules are arranged as follows: CohII-C-A-CBM-T-G-F.
Enzymes:
Xyn43A (xylanase) (T. fusca)+dockerin from C. cellulolyticum (dockerin from scaffoldin A) (designated “Xyn43A-c”)
Xyn11A (xylanase) (T. fusca)+dockerin from A. cellulolyticus (dockerin module of ScaB noted above) (designated “Xyn11A-a”)
Xyn10B (xylanase) (T. fusca)+dockerin from C. thermocellum (dockerin of Cel48S noted above) (designated “Xyn10B-t”)
Cel6A (endoglucanase) (T. fusca)+dockerin 2375 from Archaeoglobus fulgidus (designated “6A-g”)
Xyn10A (xylanase) (T. fusca)+dockerin from Ruminococcus flavefaciens (dockerin from ScaA) (designated “Xyn10A-f”)

The construction of Xyn43A-c, Xyn11A-a, Xyn10B-t and Xyn10A-f is described in Morals, S., et al., 2012, MBio, 3(6). The recombinant 6A-g was obtained by replacing CBM2 of the wild type enzyme by a dockerin from the bacterium A. fulgidus (protein source: 2375). A His-tag was added at the end of the sequence. The protein was purified using conventional Nickel beads purification protocol.

The amino acid sequence of Xyn43A-c is set forth in SEQ ID NO: 45. The dockerin module corresponds to residues 564-623 of the sequence. The polynucleotide sequence encoding Xyn43A-c is set forth in SEQ ID NO: 46.

The amino acid sequence of Xyn11A-a is set forth in SEQ ID NO: 47. The dockerin module corresponds to residues 329-399 of the sequence. The polynucleotide sequence encoding Xyn11A-a is set forth in SEQ ID NO: 48.

The amino acid sequence of Xyn10B-t is set forth in SEQ ID NO: 49. The dockerin module corresponds to residues 397-460 of the sequence. The polynucleotide sequence encoding Xyn10B-t is set forth in SEQ ID NO: 50.

The amino acid sequence of 6A-g is set forth in SEQ ID NO: 51. The polynucleotide sequence encoding 6A-g is set forth in SEQ ID NO: 52.

The amino acid sequence of Xyn10A-f is set forth in SEQ ID NO: 53. The dockerin module corresponds to residues 368-444 of the sequence. The polynucleotide sequence encoding Xyn10A-f is set forth in SEQ ID NO: 54.

Adaptor trivalent scaffold (CBM-A-B-T-DockII described above):
Scaffold:
type II dockerin module from C. thermocellum;
cohesin modules from A. cellulolyticus, B. cellulosolvens, C. thermocellum;
CBM (C. thermocellum, CBM3a of CipA);
Enzymes:
Cel9A+dockerin from A. cellulolyticus
Cel48A+dockerin from B. cellulosolvens
Cel5A+dockerin from C. thermocellum

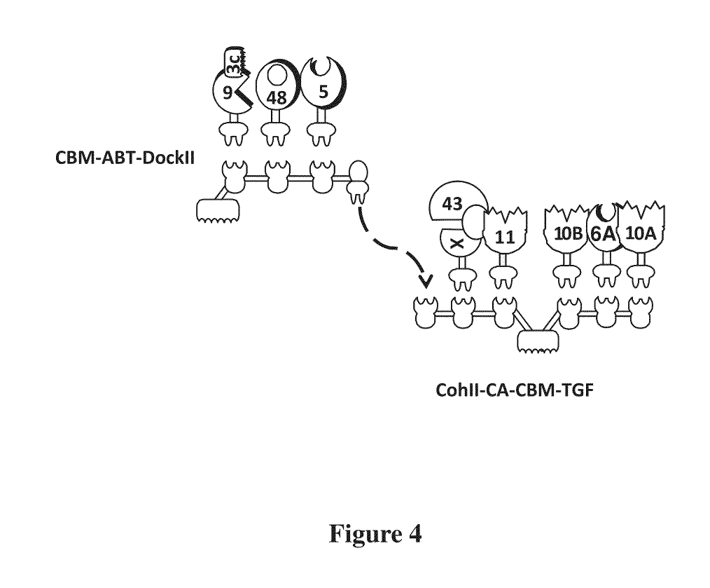

A schematic illustration of the resulting multi-enzyme complex is shown in FIG. 4.

The contribution of the attachment of the adaptor scaffold to the primary scaffold was demonstrated by using a wide variety of controls that clearly showed that the proximity between the two scaffolds is indeed important for optimized degradation of a complex cellulosic substrate.

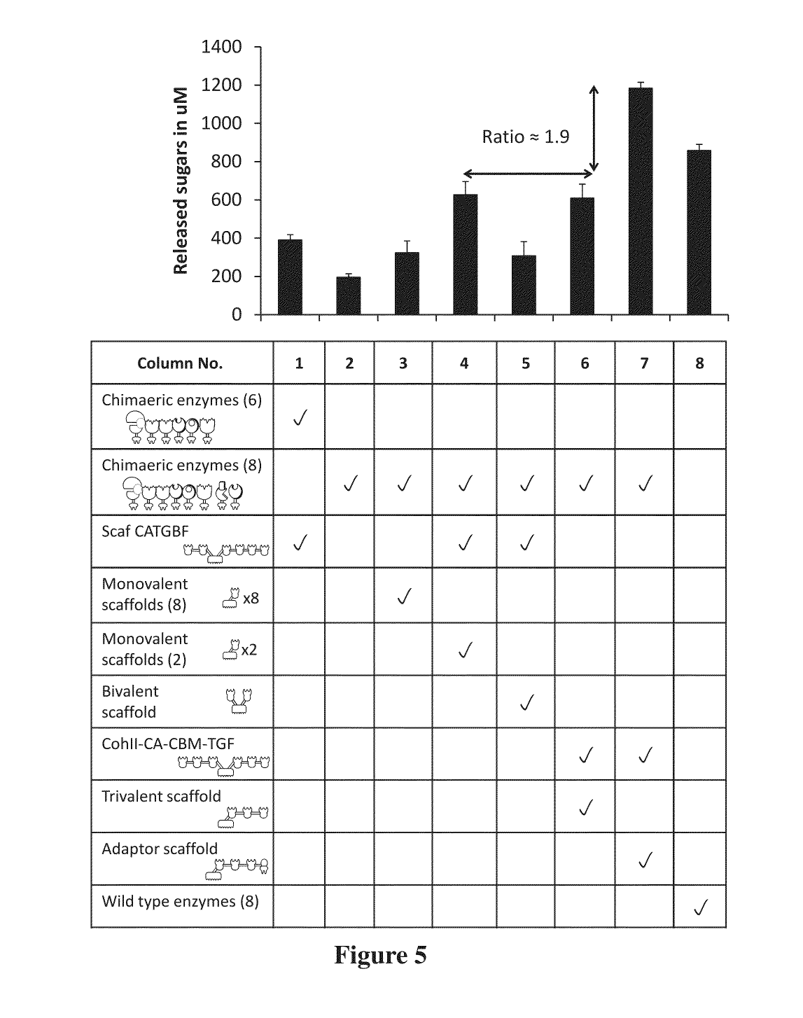

FIG. 5 presents wheat straw degradation capabilities of different chimaeric enzymatic cocktails measured as the amount of reducing sugars released after 48 hours incubation at 50° C. The experimental procedure was as in Morais et al., 2012, MBio., 3(6): e00508-12. Enzyme concentration 0.3 μM (each).

The following combinations were tested:
Complex of six recombinant enzymes bound to a hexavalent scaffold: Xyn43-c, Xyn11A-a, Xyn10B-t, Xyn10A-f, 6A-g and b-48a (Column 1 of FIG. 5).
Mixture of free eight recombinant enzymes: Xyn43-c, Xyn11A-a, Xyn10B-t, Xyn10A-f, 6A-g, b-48a, a-9A and 5A-t (Column 2 of FIG. 5).
Complexes of the eight recombinant enzymes with matching mini-scaffolds, namely, scaffold polypeptides composed of a carbohydrate binding module (CBM) and a single cohesin module, matching the dockerin module of the interacting enzyme (Column 3 of FIG. 5).
Mixture of a complex of the six recombinant enzymes Xyn43-c, Xyn11A-a, Xyn10B-t, Xyn10A-f, 6A-g and b-48a bound to a hexavalent scaffold, and complexes of a-9A and 5A-t, each bound to a mini-scaffold containing a matching cohesin (Column 4 of FIG. 5).
Mixture of a complex of the six recombinant enzymes Xyn43-c, Xyn11A-a, Xyn10B-t, Xyn10A-f, 6A-g and b-48a bound to a hexavalent scaffold, and a complex of a-9A and 5A-t bound to a bivalent scaffold containing two cohesin modules matching the dockerin modules of a-9A and 5A-t (Column 5 of FIG. 5).
Mixture of a complex of five recombinant enzymes Xyn43-c, Xyn11A-a, Xyn10B-t, 6A-g and Xyn10A-f bound to the hexavalent scaffold CohII-C-A-CBM-T-G-F (which contains one cohesin for integration with an adaptor scaffold and therefore integrates only five enzymatic subunits), and a complex of three recombinant enzymes a-9A, b-48a and 5A-9 bound to a trivalent scaffold containing cohesin modules matching the dockerin modules of the three enzymes (Column 6 of FIG. 5).
Complex of primary and adaptor scaffold with their bound enzymes: five recombinant enzymes Xyn43-c, Xyn11A-a, Xyn10B-t, 6A-g and Xyn10A-f bound to the primary scaffold CohII-C-A-CBM-T-G-F, and three recombinant enzymes a-9A, b-48a and 5A-9 bound to the adaptor scaffold CBM-A-B-T-DockII (Column 7 of FIG. 5).
Mixture of free wild-type enzymes Xyn43, Xyn11A, Xyn10B, Xyn10A, Cel6A, Cel48A, Cel9A and Cel5A (Column 8 of FIG. 5).

By comparing Columns 4 and 6 to Column 7, it is possible to observe the importance of the interaction between the primary and adaptor scaffolds. The integration of the two scaffolds resulted in a significant increase (approximately 2-fold increase) of activity compared to a mixture of non-bound primary and adaptor scaffolds (each with its enzymes). The activity was also improved compared to a hexavalent scaffold-enzyme complex mixed with monovalent scaffold-enzyme complexes (mini scaffolds).

The potency of the designer cellulosome complex was also evaluated in comparison to the extracted natural cellulosome of C. thermocellum, in the presence or absence of a betaglucosidase (BglC from T. fusca). Wheat straw degradation was tested as described in Morais et al., 2012, MBio (noted above). Incubation carried out at 50° C.

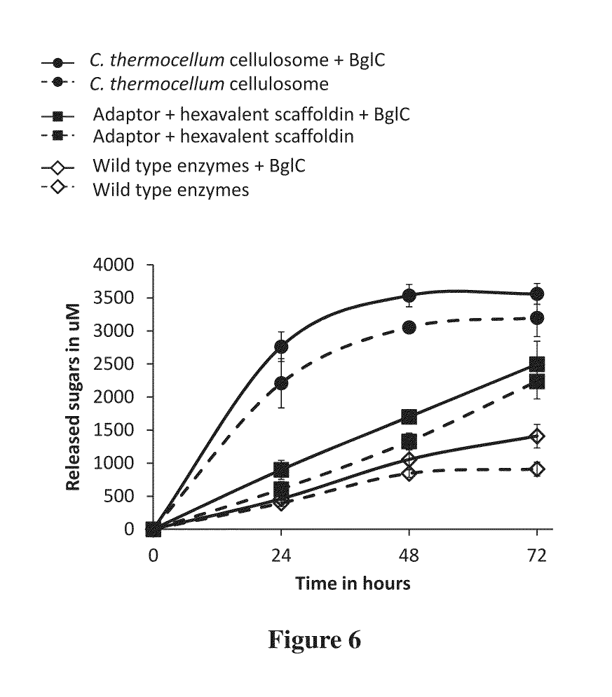

The results are summarized in FIG. 6. The designer cellulosome containing an adaptor scaffold attached to a primary scaffoldin with a total of eight chimaeric enzymes showed advantageous kinetics of degradation compared to the native cellulosome: while the activity of the C. thermocellum cellulosome appears to reach saturation after 48 hours, the designer cellulosome keeps its linear increase even after 72 hours.

In addition to the improved kinetics, further assays showed that the designer cellulosome with the bound adaptor-primary scaffolds described herein showed improved degradative capabilities compared with hitherto known designer cellulosome, for example, the designer cellulosome described in Morais et al., 2012, MBio. (noted above). The designer cellulosome described in Morais et al. is composed of a hexavalent scaffold with a total of six chimaeric enzymes, Xyn43-c, Xyn11A-a, Xyn10B-t, Xyn10A-f, g-5A and b-48a. When this hexavalent designer cellulosome was compared to the native cellulosome of C. thermocellum it showed only about 40% of the activity of the native cellulosome, while the designer cellulosome described herein showed approximately 70% of the activity of the native cellulosome (comparing wheat straw degradation in the presence of the beta-glucosidase after 72 hours of incubation).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An artificial cellulolytic multi-enzyme complex comprising:
(i) a first scaffold polypeptide comprising a plurality of cohesin modules separated by linkers comprising 5-50 amino acids, at least two of said cohesin modules having distinct binding specificities for dockerin modules, and a dockerin module;
(ii) a second scaffold polypeptide comprising a plurality of cohesin modules separated by linkers comprising 5-50 amino acids, at least two of said cohesin modules having distinct binding specificities for dockerin modules, wherein at least one of the cohesin modules has binding specificity for the dockerin of the first scaffold polypeptide; and
(iii) a plurality of carbohydrate active enzymes, each carbohydrate active enzyme comprises a dockerin module with a binding specificity for a cohesin of the first scaffold, second scaffold or both,
wherein the first and second scaffolds are bound via the dockerin of the first scaffold and the cohesin of the second scaffold having a binding specificity for said dockerin,
wherein the plurality of carbohydrate active enzymes are bound to the first and second scaffold polypeptides via dockerin-cohesin modules having mutual binding specificities, and
wherein the first scaffold, second scaffold or both further comprise a carbohydrate binding module (CBM).

2. The multi-enzyme complex of claim 1, wherein each of the first and second scaffold polypeptides comprises 3-10 cohesin modules.

3. The multi-enzyme complex of claim 2, wherein each of the first and second scaffold polypeptides comprises 3-6 cohesin modules.

4. The multi-enzyme complex of claim 1, wherein the cohesin modules originate from one or more cellulosome-producing microorganisms.

5. The multi-enzyme complex of claim 4, wherein the cellulosome-producing microorganisms are selected from the group consisting of Clostridium thermocellum, Acetivibrio cellulolyticus, Ruminococcus flavefaciens, Bacteroides cellulosolvens, Archaeoglobus fulgidus and Clostridium cellulolyticum.

6. The multi-enzyme complex of claim 1, wherein the cohesin modules originate from one or more non-cellulosomal microorganisms.

7. The multi-enzyme complex of claim 1, wherein the dockerin modules originate from one or more cellulosome-producing microorganisms.

8. The multi-enzyme complex of claim 7, wherein the cellulosome-producing microorganisms are selected from the group consisting of C. thermocellum, A. cellulolyticus, R. flavefaciens, B. cellulosolvens, A. fulgidus and C. cellulolyticum.

9. The multi-enzyme complex of claim 1, wherein the dockerin modules originate from one or more non-cellulosomal microorganisms.

10. The multi-enzyme complex of claim 1, wherein both first and second scaffold polypeptides comprise a CBM.

11. The multi-enzyme complex of claim 1, wherein the linkers are composed of 5-40 amino acids.

12. The multi-enzyme complex of claim 1, wherein the linkers are composed of 15-35 amino acids.

13. The multi-enzyme complex of claim 1, wherein the plurality of carbohydrate active enzymes comprises glycoside hydrolases, polysaccharide lyases carbohydrate esterases or combinations thereof.

14. The multi-enzyme complex of claim 13, wherein the glycoside hydrolases are selected from the group consisting of cellulases, xylanases, β-glucosidases and combinations thereof.

15. The multi-enzyme complex of claim 1, wherein the carbohydrate-active enzymes are bacterial enzymes.

16. The multi-enzyme complex of claim 15, comprising carbohydrate-active enzymes from T. fusca, C. thermocellum or both.

17. The multi-enzyme complex of claim 1, further comprising one or more scaffold polypeptides with a plurality of carbohydrate binding enzymes bound thereto, bound to the first scaffold, second scaffold or both.

18. A composition for degrading a cellulosic material comprising the multi-enzyme complex of claim 1.

19. A system for degrading a cellulosic material, the system comprising the multi enzyme complex of claim 1.

20. A method for degrading a cellulosic material, the method comprising exposing said cellulosic material to the multi-enzyme complex of claim 1.


SEQUENCE LISTING (54 sequences)

SEQ ID NO: 1 - protein, 165 aa, Clostridium thermocellum
ANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGS

SEQ ID NO: 2 - protein, 145 aa, Clostridium thermocellum
SDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNAT

SEQ ID NO: 3 - protein, 148 aa, Bacteroides cellulosolvens
SSPGNKMKIQIGDVKANQGDTVIVPITFNEVPVMGVNNCNFTLAYDKNIMEFISADAGDIVTLPMANYSYNMPSDGLVKFLYNDQAQGAMSIKEDGTFANVKFKIKQSAAFGKYSVGIKAIGSISALSNSKLIPIESIFKDGSITVTN

SEQ ID NO: 4 - protein, 146 aa, Acetivibrio cellulolyticus
GSDLQVDIGSTSGKAGSVVSVPITFTNVPKSGIYALSFRTNFDPQKVTVASIDAGSLIENASDFTTYYNNENGFASMTFEAPVDRARIIDSDGVFATINFKVSDSAKVGELYNITTNSAYTSFYYSGTDEIKNVVYNDGKIEVIAS

SEQ ID NO: 5 - protein, 29 aa, Acetivibrio cellulolyticus
PTPTQSATPTVTPSATATPTQSATPTVTP

SEQ ID NO: 6 - protein, 5 aa, Acetivibrio cellulolyticus
PTPTQ

SEQ ID NO: 7 - protein, 27 aa, Bacteroides cellulosolvens
TPTNTISVTPTNNSTPTNNSTPKPNPL

SEQ ID NO: 8 - protein, 5 aa, Bacteroides cellulosolvens
TPTNT

SEQ ID NO: 9 - protein, 35 aa, Clostridium thermocellum
PTKGATPTNTATPTKSATATPTRPSVPTNTPTNTP

SEQ ID NO: 10 - protein, 5 aa, Clostridium thermocellum
PTKGA

SEQ ID NO: 11 - protein, 31 aa, Clostridium thermocellum
VVPSTQPVTTPPATTKPPATTKPPATTIPPS

SEQ ID NO: 12 - protein, 5 aa, Clostridium thermocellum
VVPST

SEQ ID NO: 13 - protein, 721 aa, Artificial Sequence
  Polypeptide
MGPTKAPTKDGTSYKDLFLELYGKIKDPKNGYFSPDEGIPYHSIETLIVEAPDYGHVTTSEAFSYYVWLEAMYGNLTGNWSGVETAWKVMEDWIIPDSTEQPGMSSYNPNSPATYADEYEDPSYYPSELKFDTVRVGSDPVHNDLVSAYGPNMYLMHWLMDVDNWYGFGTGTRATFINTFQRGEQESTWETIPHPSIEEFKYGGPNGFLDLFTKDRSYAKQWRYTNAPDAEGRAIQAVYWANKWAKEQGKGSAVASVVSKAAKMGDFLRNDMFDKYFMKIGAQDKTPATGYDSAHYLMAWYTAWGGGIGASWAWKIGCSHAHFGYQNPFQGWVSATQSDFAPKSSNGKRDWTTSYKRQLEFYQWLQSAEGGIAGGATNSWNGRYEKYPAGTSTFYGMAYVPHPVYADPGSNQWFGFQAWSMQRVMEYYLETGDSSVKNLIKKWVDWVMSEIKLYDDGTFAIPSDLEWSGQPDTWTGTYTGNPNLHVRVTSYGTDLGVAGSLANALATYAAATERWEGKLDTKARDMAAELVNRAWYNFYCSEGKGVVTEEARADYKRFFEQEVYVPAGWSGTMPNGDKIQPGIKFIDIRTKYRQDPYYDIVYQAYLRGEAPVLNYHRFWHEVDLAVAMGVLATYFPDMTYKVPGTPSTKLYGDVNDDGKVNSTDAVALKRYVLRSGISINTDNADLNEDGRVNSTDLGILKRYILKEIDTLPYKNHHHHHH

SEQ ID NO: 14 - DNA, 2166 bp, Artificial Sequence
  Polynucleotide
atgggtcctacaaaggcacctacaaaagatgggacatcttataaggatcttttccttgaactctacggaaaaattaaagatcctaagaacggatatttcagcccagacgagggaattccttatcactcaattgaaacattgatcgttgaagcgccggactacggtcacgttactaccagtgaggctttcagctattatgtatggcttgaagcaatgtatggaaatctcacaggcaactggtccggagtagaaacagcatggaaagttatggaggattggataattcctgacagcacagagcagccgggtatgtcttcttacaatccaaacagccctgccacatatgctgacgaatatgaggatccttcatactatccttcagagttgaagtttgataccgtaagagttggatccgaccctgtacacaacgaccttgtatccgcatacggtcctaacatgtacctcatgcactggttgatggacgttgacaactggtacggttttggtacaggaacacgggcaacattcataaacaccttccaaagaggtgaacaggaatccacatgggaaaccattcctcatccgtcaatagaagagttcaaatacggcggaccgaacggattccttgatttgtttacaaaggacagatcatatgcaaaacagtggcgttatacaaacgctcctgacgcagaaggccgtgctatacaggctgtttactgggcaaacaaatgggcaaaggagcagggtaaaggttctgccgttgcttccgttgtatccaaggctgcaaagatgggtgacttcttgagaaacgacatgttcgacaaatacttcatgaagatcggtgcacaggacaagactcctgctaccggttatgacagtgcacactaccttatggcctggtatactgcatggggtggtggaattggtgcatcctgggcatggaagatcggatgcagccacgcacacttcggatatcagaacccattccagggatgggtaagtgcaacacagagcgactttgctcctaaatcatccaacggtaagagagactggacaacaagctacaagagacagcttgaattctatcagtggttgcagtcggctgaaggtggtattgccggtggagcaaccaactcctggaacggtagatatgagaaatatcctgctggtacgtcaacgttctatggtatggcatatgttccgcatcctgtatacgctgacccgggtagtaaccagtggttcggattccaggcatggtcaatgcagcgtgtaatggagtactacctcgaaacaggagattcatcagttaagaatttgattaagaagtgggtcgactgggtaatgagcgaaattaagctctatgacgatggaacatttgcaattcctagcgacctcgagtggtcaggtcagcctgatacatggaccggaacatacacaggcaacccgaacctccatgtaagagtaacttcttacggtactgaccttggtgttgcaggttcacttgcaaatgctcttgcaacttatgccgcagctacagaaagatgggaaggaaaacttgatacaaaagcaagagacatggctgctgaactggttaaccgtgcatggtacaacttctactgctctgaaggaaaaggtgttgttactgaggaagcacgtgctgactacaaacgtttctttgagcaggaagtatacgttccggcaggttggagcggtactatgccgaacggtgacaagattcagcctggtattaagttcatagacatccgtacaaaatatagacaagatccttactacgatatagtatatcaggcatacttgagaggcgaagctcctgtattgaattatcaccgcttctggcatgaagttgaccttgcagttgcaatgggtgtattggctacatacttcccggatatgacatataaagtacctggtactccttctactaaattatacggcgacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctcaaagaaatagatacattgccgtacaagaaccaccatcaccatcaccattaa

SEQ ID NO: 15 - protein, 466 aa, Artificial Sequence
  Polypeptide
GVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLVPPKGTATVLYGDVDNDGNVDSDDYAYMRQWLIGMIADFPGGDIGLANADVDGDGNVDSDDYAYMRQWLIGMISEFPAEQKALEHHHHHH

SEQ ID NO: 16 - DNA, 1404 bp, Artificial Sequence
  Polynucleotide
atgggtgtgccttttaacacaaaatacccctatggtcctacttctattgccgataatcagtcggaagtaactgcaatgctcaaagcagaatgggaagactggaagagcaagagaattacctcgaacggtgcaggaggatacaagagagtacagcgtgatgcttccaccaattatgatacggtatccgaaggtatgggatacggacttcttttggcggtttgctttaacgaacaggctttgtttgacgatttataccgttacgtaaaatctcatttcaatggaaacggacttatgcactggcacattgatgccaacaacaatgttacaagtcatgacggcggcgacggtgcggcaaccgatgctgatgaggatattgcacttgcgctcatatttgcggacaagttatggggttcttccggtgcaataaactacgggcaggaagcaaggacattgataaacaatctttacaaccattgtgtagagcatggatcctatgtattaaagcccggtgacagatggggaggttcatcagtaacaaacccgtcatattttgcgcctgcatggtacaaagtgtatgctcaatatacaggagacacaaggtggaatcaagtggcggacaagtgttaccaaattgttgaagaagttaagaaatacaacaacggaaccggccttgttcctgactggtgtactgcaagcggaactccggcaagcggtcagagttacgactacaaatatgatgctacacgttacggctggagaactgccgtggactattcatggtttggtgaccagagagcaaaggcaaactgcgatatgctgaccaaattctttgccagagacggggcaaaaggaatcgttgacggatacacaattcaaggttcaaaaattagcaacaatcacaacgcatcatttataggacctgttgcggcagcaagtatgacaggttacgatttgaactttgcaaaggaactttatagggagactgttgctgtaaaggacagtgaatattacggatattacggaaacagcttgagactgctcactttgttgtacataacaggaaacttcccgaatcctttgagtgacctttccggccaaccgacaccaccgtcgaatccgacaccttcattggtacctccaaaaggcacagctacagtattatatggtgacgttgataatgatggaaatgttgattcagacgactatgcatatatgagacaatggttgatcggtatgattgctgatttccctggaggagatatcggattagctaatgctgatgttgatggagacggaaatgtagattcagatgactatgcgtacatgagacaatggttaataggaatgatttccgagttcccagcagaacaaaaagcgctcgagcaccaccaccaccaccactga

SEQ ID NO: 17 - protein, 878 aa, Artificial Sequence
  Polypeptide
MGHHHHHHLEDKSPKLPDYKNDLLYERTFDEGLCFPWHTCEDSGGKCDFAVVDVPGEPGNKAFRLTVIDKGQNKWSVQMRHRGITLEQGHTYTVRFTIWSDKSCRVYAKIGQMGEPYTEYWNNNWNPFNLTPGQKLTVEQNFTMNYPTDDTCEFTFHLGGELAAGTPYYVYLDDVSLYDPRFVKPVEYVLPQPDVRVNQVGYLPFAKKYATVVSSSTSPLKWQLLNSANQVVLEGNTIPKGLDKDSQDYVHWIDFSNFKTEGKGYYFKLPTVNSDTNYSHPFDISADIYSKMKFDALAFFYHKRSGIPIEMPYAGGEQWTRPAGHIGVAPNKGDTNVPTWPQDDEYAGRPQKYYTKDVTGGWYDAGDHGKYVVNGGIAVWTLMNMYERAKIRGIANQGAYKDGGMNIPERNNGYPDILDEARWEIEFFKKMQVTEKEDPSIAGMVHHKIHDFRWTALGMLPHEDPQPRYLRPVSTAATLNFAATLAQSARLWKDYDPTFAADCLEKAEIAWQAALKHPDIYAEYTPGSGGPGGGPYNDDYVGDEFYWAACELYVTTGKDEYKNYLMNSPHYLEMPAKMGENGGANGEDNGLWGCFTWGTTQGLGTITLALVENGLPSADIQKARNNIAKAADKWLENIEEQGYRLPIKQAEDERGGYPWGSNSFILNQMIVMGYAYDFTGNSKYLDGMQDGMSYLLGRNGLDQSYVTGYGERPLQNPHDRFWTPQTSKKFPAPPPGIIAGGPNSRFEDPTITAAVKKDTPPQKCYIDHTDSWSTNEITINWNAPFAWVTAYLDEIDLITPPGTKFIYGDVDGNGSVRINDAVLIRDYVLGKINEFPYEYGMLAADVDGNGSIKINDAVLVRDYVLGKIFLFPVEEKEE

SEQ ID NO: 18 - DNA, 2637 bp, Artificial Sequence
  Polynucleotide
atgggccatcaccatcaccatcacttagaagacaagtctccaaagttgccggattataaaaacgaccttttgtatgaaagaacattcgacgaaggtctttgctttccgtggcatacttgcgaagacagtggaggaaaatgtgatttcgctgttgttgatgttccaggagagcctgggaacaaagctttccgcttgacagtaattgacaaaggacaaaacaagtggagtgtccagatgagacacagaggtattaccctcgagcaaggacatacatacacggtaaggtttacgatttggtctgacaaatcctgtagggtttatgctaaaattggtcagatgggtgaaccctatactgaatattggaacaataactggaatccattcaaccttacaccaggacagaagcttacagttgaacagaattttacaatgaactatcctactgatgacacatgcgagttcacattccatttgggtggagaacttgctgcaggtacaccttactatgtttaccttgatgatgtatctctctacgatcctaggtttgtaaagcctgttgaatatgtacttccgcagccggatgtacgtgttaaccaggtaggatacttaccgtttgcaaagaagtatgctactgttgtatcttcttcaaccagcccgcttaagtggcagcttctcaattcggcaaatcaggttgttttggaaggtaatacaataccaaaaggacttgacaaagattcacaggattatgtacattggatagatttctccaactttaagactgaaggaaaaggttattacttcaagcttccgactgtaaacagcgatacaaattacagccatcctttcgatatcagtgctgatatttactccaagatgaaatttgatgcattggcattcttctatcacaagagaagcggtattcctattgaaatgccgtatgcaggaggagaacagtggaccagacctgcaggacatattggtgttgctccgaacaaaggagacacaaatgttcctacatggcctcaggatgatgaatatgcaggaagacctcaaaaatattatacaaaagatgtaaccggtggatggtatgatgccggtgaccacggtaaatatgttgtaaacggcggtatagctgtttggacattgatgaacatgtatgaaagggcaaaaatcagaggcatagctaatcaaggtgcttataaagacggtggaatgaacataccggagagaaataacggttatccggacattcttgatgaagcaagatgggaaattgagttctttaagaaaatgcaggtaactgaaaaagaggatccttccatagccggaatggtacaccacaaaattcacgacttcagatggactgctttgggtatgttgcctcacgaagatccccagccacgttacttaaggccggtaagtacggctgcgactttgaactttgcggcaactttggcacaaagtgcacgtctttggaaagattatgatccgacttttgctgctgactgtttggaaaaggctgaaatagcatggcaggcggcattaaagcatcctgatatttatgctgagtatactcccggtagcggtggtcccggaggcggaccatacaatgacgactatgtcggagacgaattctactgggcagcctgcgaactttatgtaacaacaggaaaagacgaatataagaattacctgatgaattcacctcactatcttgaaatgcctgcaaagatgggtgaaaacggtggagcaaacggagaagacaacggattgtggggatgcttcacctggggaactactcaaggattgggaactattactcttgcattagttgaaaacggattgccgtctgcagacattcaaaaggcaagaaacaatatagctaaagctgcagacaaatggcttgagaatattgaagagcaaggttacagactgccgatcaaacaggcggaggatgagagaggcggttatccatggggttcaaactccttcattttgaaccagatgatagttatgggatacgcatatgactttacaggcaacagcaagtatcttgacggaatgcaggatggtatgagctacctgttgggaagaaacggactggatcagtcctatgtaacagggtatggtgagcgtccacttcagaatcctcatgacagattctggacgccacagacaagtaagaaattccctgctccacctccgggtataattgccggtggtccgaactcccgtttcgaagacccgacaataactgcagcagttaagaaggatacaccgccgcagaagtgctacattgaccatacagactcatggtcaaccaacgagataactattaactggaatgctccgtttgcatgggttacagcttatctcgatgaaattgacttaataacaccgccaggtaccaaatttatatatggtgatgttgatggtaatggaagtgtaagaattaatgatgctgtcctaataagagactatgtattaggaaaaatcaatgaattcccatatgaatatggtatgcttgcagcagatgttgatggtaatggaagtataaaaattaatgatgctgttctagtaagagactacgtgttaggaaagatatttttattccctgttgaagagaaagaagaataa

SEQ ID NO: 19 - DNA, 29 bp, Artificial Sequence
  Primer
cagtccatgggtcctacaaaggcacctac

SEQ ID NO: 20 - DNA, 32 bp, Artificial Sequence
  Primer
cgcgaagcttttaatggtgatggtgatggtgg

SEQ ID NO: 21 - DNA, 29 bp, Artificial Sequence
  Primer
cagtccatgggtgtgccttttaacacaaa

SEQ ID NO: 22 - DNA, 30 bp, Artificial Sequence
  Primer
cacgctcgagataaggtaggtggggtatgc

SEQ ID NO: 23 - DNA, 80 bp, Artificial Sequence
  Primer
gtttaactttaagaaggagatataccatgggccatcaccatcaccatcacttagaagacaagtctccaaagttgccggat

SEQ ID NO: 24 - DNA, 64 bp, Artificial Sequence
  Primer
gagtgcggccgcaagcttgtcgacggagctcttatttatgtggcaatacatctatctctttaag

SEQ ID NO: 25 - DNA, 67 bp, Artificial Sequence
  Primer
ctcgatgaaattgacttaataacaccgccaggtaccaaatttatatatggtgatgttgatggtaatg

SEQ ID NO: 26 - DNA, 68 bp, Artificial Sequence
  Primer
gagtgcggccgcaagcttgtcgacggagctcttattcttctttctcttcaacagggaataaaaatatc

SEQ ID NO: 27 - DNA, 35 bp, Artificial Sequence
  Primer
attcaaccatgggtgtgccttttaacacaaaatac

SEQ ID NO: 28 - DNA, 46 bp, Artificial Sequence
  Primer
atattgctcgagtaatgtggtaccaatgaaggtgtcggattcgacg

SEQ ID NO: 29 - DNA, 30 bp, Artificial Sequence
  Primer
actttaggtacctccaaaaggcacagctac

SEQ ID NO: 30 - DNA, 30 bp, Artificial Sequence
  Primer
attaatctcgagcgctttttgttctgctgg

SEQ ID NO: 31 - protein, 866 aa, Artificial Sequence
  Polypeptide
MANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSGSDLQVDIGSTSGKAGSVVSVPITFTNVPKSGIYALSFRTNFDPQKVTVASIDAGSLIENASDFTTYYNNENGFASMTFEAPVDRARIIDSDGVFATINFKVSDSAKVGELYNITTNSAYTSFYYSGTDEIKNVVYNDGKIEVIASPTPTQSATPTVTPSATATPTQSATPTVTPSSPGNKMKIQIGDVKANQGDTVIVPITFNEVPVMGVNNCNFTLAYDKNIMEFISADAGDIVTLPMANYSYNMPSDGLVKFLYNDQAQGAMSIKEDGTFANVKFKIKQSAAFGKYSVGIKAIGSISALSNSKLIPIESIFKDGSITVTNTPTNTISVTPTNNSTPTNNSTPKPNPLSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATRSTNKPVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDYDAQLEHHHHHH

SEQ ID NO: 32 - DNA, 2601 bp, Artificial Sequence
  Polynucleotide
atggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcaggatccgatttacaggttgacattggaagtactagtggaaaagcaggtagtgttgttagtgtacctataacatttactaatgtacctaaatcaggtatctatgctctaagttttagaacaaatttcgacccacaaaaggtaactgtagcaagtatagatgctggctcactgattgaaaatgcttctgattttactacttattataataatgaaaatggttttgcatcaatgacgtttgaagccccagttgatagagctagaatcatagatagtgatggtgtatttgcaaccattaactttaaagttagtgatagtgccaaagtaggtgaactttacaatattactactaatagtgcatatacttcattctattattctggaactgatgaaatcaaaaatgttgtttacaatgatggaaaaattgaggtaattgcaagtcctaccccgacgcaatcagccactccaacggtaactccttcagccaccgcgacgcctacccagagtgctacgccgactgtaacgccaagttcaccaggaaataaaatgaaaattcaaattggtgatgtaaaagctaatcagggagatacagttatagtacctataactttcaatgaagttcctgtaatgggtgttaataactgtaatttcactttagcttatgacaaaaatattatggaatttatctctgctgatgcaggtgatattgtaacattgccaatggctaactatagctacaatatgccatctgatgggctagtaaaatttttatataatgatcaagctcaaggtgcaatgtcaataaaagaagatggtacttttgctaatgttaaatttaaaattaagcagagtgccgcatttgggaaatattcagtaggcatcaaagcaattggttcaatttccgctttaagcaatagtaagttaatacctattgaatcaatatttaaagatggaagcattactgtaactaatacgccgaccaatactatcagtgttactccgacaaacaattcgactcctacgaataacagtacgccaaagccaaacccgttatccgacggtgtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgttggcaatgcaacaagatccactaataaacctgtaatagaaggatataaagtatccggatacattttgccagacttctccttcgacgctactgttgcaccacttgtaaaggccggattcaaagttgaaatagtaggaacagaattgtatgcagtaacagatgcaaacggatactttgaaataaccggagtacctgcaaatgcaagcggatatacattgaagatttcaagagcaacttacttggacagagtaattgcaaatgttgtagtaacgggagatacttcagtttcaacttcacaggctccaataatgatgtgggtaggagacatagtgaaagacaattctatcaacctgttggacgttgcagaagttatccgttgcttcaacgctactaaaggaagcgcaaactacgtagaagaacttgacattaatagaaacggcgcaattaacatgcaagacataatgattgttcataagcactttggagctacatcaagtgattacgacgcacagctcgagcaccaccaccaccaccactga

SEQ ID NO: 33 - protein, 712 aa, Artificial Sequence
  Polypeptide
MTHHHHHHAMAKFIYGDVDGNGSVRINDAVLIRDYVLGKINEFPYEYGMLAADVDGNGSIKINDAVLVRDYVLGKIFLFPVEEKEEVPPLATGTAHAEPAFNYAEALQKSMFFYEAQRSGKLPENNRVSWRGDSGLNDGADVGLDLTGGWYDAGDHVKFGFPMAFTATMLAWGAIESPEGYIRSGQMPYLKDNLRWVNDYFIKAHPSPNVLYVQVGDGDADHKWWGPAEVMPMERPSFKVDPSCPGSDVAAETAAAMAASSIVFADDDPAYAATLVQHAKQLYTFADTYRGVYSDCVPAGAFYNSWSGYQDELVWGAYWLYKATGDDSYLAKAEYEYDFLSTEQQTDLRSYRWTIAWDDKSYGTYVLLAKETGKQKYIDDANRWLDYWTVGVNGQRVPYSPGGMAVLDTWGALRYAANTAFVALVYAKVIDDPVRKQRYHDFAVRQINYALGDNPRNSSYVVGFGNNPPRNPHHRTAHGSWTDSIASPAENRHVLYGALVGGPGSPNDAYTDDRQDYVANEVATDYNAGFSSALAMLVEEYGGTPLADFPPTEEPDGPEIFVEAQINTPGTTFTEIKAMIRNQSGWPARMLDKGTFRYWFTLDEGVDPADITVSSAYNQCATPEDVHHVSGDLYYVEIDCTGEKIFPGGQSEHRREVQFRIAGGPGWDPSNDWSFQGIGNELAPAPYIVLYDDGVPVWGTAPEEGEEPGGGE

SEQ ID NO: 34 - DNA, 2148 bp, Artificial Sequence
  Polynucleotide
atgacccatcaccatcaccatcacgccatggctaaatttatatatggtgatgttgatggtaatggaagtgtaagaattaatgatgctgtcctaataagagactatgtattaggaaaaatcaatgaattcccatatgaatatggtatgcttgcagcagatgttgatggtaatggaagtataaaaattaatgatgctgttctagtaagagactacgtgttaggaaagatatttttattccctgttgaagagaaagaagaggtaccccccttggccacgggaaccgcccacgccgaaccggcgttcaactacgccgaagccctccagaagtcgatgttcttctacgaggcccaacgctccgggaaactcccggagaacaaccgggtctcctggcgcggcgactccgggctcaacgacggcgcggacgtgggactcgacctcaccggcggctggtacgacgccggcgaccacgtgaaattcggcttccccatggccttcaccgcgaccatgctcgcctggggcgccatcgaaagcccggaaggctacatccgctccggccagatgccctacctcaaggacaacctgcgctgggtcaacgactacttcatcaaagcccacccctcgcccaacgtgctgtacgtgcaggtcggcgacggcgacgccgaccacaagtggtggggtccggccgaagtcatgccgatggagcggcccagcttcaaagtggacccctcctgcccgggcagcgacgtcgcagccgaaaccgccgcggccatggccgcgtcctccatcgtgttcgccgacgacgaccctgcgtacgcggccaccctcgtgcagcacgccaagcagctctacacgttcgccgacacctaccgcggcgtgtactccgactgcgtgcccgccggagcgttctacaactcctggtcgggctaccaggacgagctcgtctggggcgcctactggctgtacaaggccaccggggacgactcctacttggcgaaggccgagtacgagtacgacttcctctccaccgagcagcagaccgacctccgcagctaccggtggaccatcgcctgggacgacaagtcctacggcacctacgtgctgctcgccaaggaaaccggcaagcaaaaatacatcgacgacgccaaccggtggctcgactactggacggtcggcgtcaacggccagcgcgtgccctactcccccggcgggatggctgtgctcgacacctggggagccctgcgctacgccgctaacaccgcgttcgtcgccctcgtctacgccaaggtgatcgacgaccccgtccgcaagcagcgataccacgacttcgcggtgcggcagatcaactacgcgctcggcgacaacccgcggaactccagctacgtggtgggcttcggcaacaacccgccgcgcaacccccaccaccgcaccgcgcacgggtcgtggaccgacagcatcgcctcgcccgcggagaaccggcacgtcctctacggcgccctcgtcggcggtcccggctccccgaacgacgcctacaccgacgaccggcaggactacgtcgccaacgaagtcgccaccgactacaacgccggattctccagcgcgctggccatgctggtcgaagagtacggcggcaccccgctggcggacttcccgcccaccgaggagcccgacggaccggagatcttcgtggaagcccagatcaacacgccgggcaccacgttcaccgagatcaaagccatgatccgcaaccagtcgggctggccggcccggatgctggacaagggcaccttccggtactggttcaccctcgatgaaggcgtggaccccgcggacatcacggtgagctccgcctacaaccagtgcgccaccccggaggacgtccaccacgtctccggcgacctgtactacgtggagatcgactgcaccggggagaagatcttccccggcggccagtcggagcaccgccgcgaagtccagttccgcatcgccggcggccccggatgggacccctccaacgactggtccttccaaggcatcggcaacgaactcgcccccgccccgtacatcgtgctctacgacgacggtgtaccggtgtggggcaccgcccccgaggaaggggaagagcccggcggcggagaataactcgagtga

SEQ ID NO: 35 - protein, 749 aa, Artificial Sequence
  Polypeptide
MAHHHHHHPKGTATVLYGDVDNDGNVDSDDYAYMRQWLIGMIADFPGGDIGLANADVDGDGNVDSDDYAYMRQWLIGMISEFPAEQKAVPGHDSAEVTVREIDPNTSSYDQAFLEQYEKIKDPASGYFREFNGLLVPYHSVETMIVEAPDHGHQTTSEAFSYYLWLEAYYGRVTGDWKPLHDAWESMETFIIPGTKDQPTNSAYNPNSPATYIPEQPNADGYPSPLMNNVPVGQDPLAQELSSTYGTNEIYGMHWLLDVDNVYGFGFCGDGTDDAPAYINTYQRGARESVWETIPHPSCDDFTHGGPNGYLDLFTDDQNYAKQWRYTNAPDADARAVQVMFWAHEWAKEQGKENEIAGLMDKASKMGDYLRYAMFDKYFKKIGNCVGATSCPGGQGKDSAHYLLSWYYSWGGSLDTSSAWAWRIGSSSSHQGYQNVLAAYALSQVPELQPDSPTGVQDWATSFDRQLEFLQWLQSAEGGIAGGATNSWKGSYDTPPTGLSQFYGMYYDWQPVWNDPPSNNWFGFQVWNMERVAQLYYVTGDARAEAILDKWVPWAIQHTDVDADNGGQNFQVPSDLEWSGQPDTWTGTYTGNPNLHVQVVSYSQDVGVTAALAKTLMYYAKRSGDTTALATAEGLLDALLAHRDSIGIATPEQPSWDRLDDPWDGSEGLYVPPGWSGTMPNGDRIEPGATFLSIRSFYKNDPLWPQVEAHLNDPQNVPAPIVERHRFWAQVEIATAFAAHDELFGAGAP

SEQ ID NO: 36 - DNA, 2250 bp, Artificial Sequence
  Polynucleotide
atggcccaccatcaccatcaccatccaaaaggcacagctacagtattatatggtgacgttgataatgatggaaatgttgattcagacgactatgcatatatgagacaatggttgatcggtatgattgctgatttccctggaggagatatcggattagctaatgctgatgttgatggagacggaaatgtagattcagatgactatgcgtacatgagacaatggttaataggaatgatttccgagttcccagcagaacaaaaagcggtacccggccacgactcggccgaggtgacggtccgggagatcgacccgaacaccagctcctacgaccaggccttcctggagcagtacgagaagatcaaggaccccgccagcggctacttccgcgaattcaacgggctcctggtcccctaccactcggtggagaccatgatcgtcgaggctccggaccacggccaccagaccacgtccgaggcgttcagctactacctgtggctggaggcgtactacggccgggtcaccggtgactggaagccgctccacgacgcctgggagtcgatggagaccttcatcatccccggcaccaaggaccagccgaccaactccgcctacaacccgaactccccggcgacctacatccccgagcagcccaacgctgacggctacccgtcgcctctcatgaacaacgtcccggtgggtcaagacccgctcgcccaggagctgagctccacctacgggaccaacgagatctacggcatgcactggctgctcgacgtggacaacgtctacggcttcgggttctgcggcgacggcaccgacgacgcccccgcctacatcaacacctaccagcgtggtgcgcgcgagtcggtgtgggagaccattccgcacccgtcctgcgacgacttcacgcacggcggccccaacggctacctggacctgttcaccgacgaccagaactacgccaagcagtggcgctacaccaacgcccccgacgctgacgcgcgggccgtccaggtgatgttctgggcgcacgaatgggccaaggagcagggcaaggagaacgagatcgcgggcctgatggacaaggcgtccaagatgggcgactacctccggtacgcgatgttcgacaagtacttcaagaagatcggcaactgcgtcggcgccacctcctgcccgggtggccaaggcaaggacagcgcgcactacctgctgtcctggtactactcctggggcggctcgctcgacacctcctctgcgtgggcgtggcgtatcggctccagctcctcgcaccagggctaccagaacgtgctcgctgcctacgcgctctcgcaggtgcccgaactgcagcctgactccccgaccggtgtccaggactgggccaccagcttcgaccgccagttggagttcctccagtggctgcagtccgctgaaggtggtatcgccggtggcgccaccaacagctggaagggaagctacgacaccccgccgaccggcctgtcgcagttctacggcatgtactacgactggcagccggtctggaacgacccgccgtccaacaactggttcggcttccaggtctggaacatggagcgcgtcgcccagctctactacgtgaccggcgacgcccgggccgaggccatcctcgacaagtgggtgccgtgggccatccagcacaccgacgtggacgccgacaacggcggccagaacttccaggtcccctccgacctggagtggtcgggccagcctgacacctggaccggcacctacaccggcaacccgaacctgcacgtccaggtcgtctcctacagccaggacgtcggtgtgaccgccgctctggccaagaccctgatgtactacgcgaagcgttcgggcgacaccaccgccctcgccaccgcggagggtctgctggacgccctgctggcccaccgggacagcatcggtatcgccacccccgagcagccgagctgggaccgtctggacgacccgtgggacggctccgagggcctgtacgtgccgccgggctggtcgggcaccatgcccaacggtgaccgcatcgagccgggcgcgaccttcctgtccatccgctcgttctacaagaacgacccgctgtggccgcaggtcgaggcacacctgaacgacccgcagaacgtcccggcgccgatcgtggagcgccaccgcttctgggctcaggtggaaatcgcgaccgcgttcgcagcccacgacgaactgttcggggccggagctccctga

SEQ ID NO: 37 - protein, 384 aa, Artificial Sequence
  Polypeptide
MVERYGKVQVCGTQLCDEHGNPVQLRGMSTHGIQWFDHCLTDSSLDALAYDWKADIIRLSMYIQEDGYETNPRGFTDRMHQLIDMATARGLYVIVDWHILTPGDPHYNLDRAKTFFAEIAQRHASKTNVLYEIANEPNGVSWASIKSYAEEVIPVIRQRDPDSVIIVGTRGWSSLGVSEGSGPAEIAANPVNASNIMYAFHFYAASHRDNYLNALREASELFPVFVTEFGTETYTGDGANDFQMADRYIDLMAERKIGWTKWNYSDDFRSGAVFQPGTCASGGPWSGSSLKASGQWVRSKLQSVPESSSTGLGDLNGDGNINSSDLQALKRHLLGISPLTGEALLRADVNRSGKVDSTDYSVLKRYILRIITEFPGLEHHHHHH

SEQ ID NO: 38 - DNA, 1155 bp, Artificial Sequence
  Polynucleotide
atggtcgagcggtacggcaaagtccaggtctgcggcacccagctctgcgacgagcacggcaacccggtccaactgcgcggcatgagcacccacggcatccagtggttcgaccactgcctgaccgacagctcgctggacgccctggcctacgactggaaggccgacatcatccgcctgtccatgtacatccaggaagacggctacgagaccaacccgcgcggcttcaccgaccggatgcaccagctcatcgacatggccacggcgcgcggcctgtacgtgatcgtggactggcacatcctcaccccgggcgatccccactacaacctggaccgggccaagaccttcttcgcggaaatcgcccagcgccacgccagcaagaccaacgtgctctacgagatcgccaacgaacccaacggagtgagctgggcctccatcaagagctacgccgaagaggtcatcccggtgatccgccagcgcgaccccgactcggtgatcatcgtgggcacccgcggctggtcgtcgctcggcgtctccgaaggctccggccccgccgagatcgcggccaacccggtcaacgcctccaacatcatgtacgccttccacttctacgcggcctcgcaccgcgacaactacctcaacgcgctgcgtgaggcctccgagctgttcccggtcttcgtcaccgagttcggcaccgagacctacaccggtgacggcgccaacgacttccagatggccgaccgctacatcgacctgatggcggaacggaagatcgggtggaccaagtggaactactcggacgacttccgttccggcgcggtcttccagccgggcacctgcgcgtccggcggcccgtggagcggttcgtcgctgaaggcgtccggacagtgggtgcggagcaagctccagtcggtacctgaaagcagttccacaggtctgggggatttaaatggtgacggaaatattaactcgtcggaccttcaggcgttaaagaggcatttgctcggtatatcaccgcttacgggagaggctcttttaagagcggatgtaaataggagcggcaaagtggattctactgactattcagtgctgaaaagatatatactccgcattattacagagttccccggactcgagcaccaccaccaccaccactga

SEQ ID NO: 39 - protein, 165 aa, Clostridium cellulolyticum
DSLKVTVGTANGKPGDTVTVPVTFADVAKMKNVGTCNFYLGYDASLLEVVSVDAGPIVKNAAVNFSSSASNGTISFLFLDNTITDELITADGVFANIKFKLKSVTAKTTTPVTFKDGGAFGDGTMSKIASVTKTNGSVTIDPTKGATPTNTATPTKSATATPTRP

SEQ ID NO: 40 - protein, 158 aa, Archaeoglobus fulgidus
VPPKTTIIAGSAEAPQGSDIQVPVKIENADKVGSINLILSYPNVLEVEDVLQGSLTQNSLFDYNVEGNQIKVGIADSNGISGDGSLFYVKFRVTGNEKAEQAENVKGKLRGLGQQLSEITLRNSHALTLQGIEIYDIDGNSVKVATINGTFRIVSQEE

SEQ ID NO: 41 - protein, 126 aa, Ruminococcus flavefaciens
GTVEWLIPTVTAAPGQTVTMPVVVKSSSLAVAGAQFKIQAATGVRYSSKTDGDAYGSGIVYNNSKYAFGQGAGRGIVAADDSVVLTLAYTVPADCAEGTYDVKWSDAFVSDTDGQNITSKVTLTDG

SEQ ID NO: 42 - protein, 202 aa, Clostridium thermocellum
VALELDKTKVKVGDIITATIKIENMKNFAGYQLNIKYDPTMLEAIELETGSAIAKRTWPVTGGTVLQSDNYGKTTAVANDVGAGIINFAEAYSNLTKYRETGVAEETGIIGKIGFRVLKAGSTAIRFEDTTAMPGAIEGTYMFDWYGENIKGYSVVQPGEIVVEGEEPGEEPTEEPVPTETSVDPTPTVTEEPVPSELPDSY

SEQ ID NO: 43 - protein, 1190 aa, Artificial Sequence
  Polypeptide
MGVALELDKTKVKVGDIITATIKIENMKNFAGYQLNIKYDPTMLEAIELETGSAIAKRTWPVTGGTVLQSDNYGKTTAVANDVGAGIINFAEAYSNLTKYRETGVAEETGIIGKIGFRVLKAGSTAIRFEDTTAMPGAIEGTYMFDWYGENIKGYSVVQPGEIVVEGEEPGEEPTEEPVPTETSVDPTPTVTEEPVPSELPDSYARLKVTVGTANGKPGDTVTVPVTFADVAKMKNVGTCNFYLGYDASLLEVVSVDAGPIVKNAAVNFSSSASNGTISFLFLDNTITDELITADGVFANIKFKLKSVTAKTTTPVTFKDGGAFGDGTMSKIASVTKTNGSVTIDPTKGATPTNTATPTKSATATPTRPSVPRPHLQVDIGSTSGKAGSVVSVPITFTNVPKSGIYALSFRTNFDPQKVTVASIDAGSLIENASDFTTYYNNENGFASMTFEAPVDRARIIDSDGVFATINFKVSDSAKVGELYNITTNSAYTSFYYSGTDEIKNVVYNDGKIEVIASVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDIGSVPPKTTIIAGSAEAPQGSDIQVPVKIENADKVGSINLILSYPNVLEVEDVLQGSLTQNSLFDYNVEGNQIKVGIADSNGISGDGSLFYVKFRVTGNEKAEQAENVKGKLRGLGQQLSEITLRNSHALTLQGIEIYDIDGNSVKVATINGTFRIVSQEEASAGGLSAVQPNVSLGEVLDVSANRTAADGTVEWLIPTVTAAPGQTVTMPVVVKSSSLAVAGAQFKIQAATGVRYSSKTDGDAYGSGIVYNNSKYAFGQGAGRGIVAADDSVVLTLAYTVPADCAEGTYDVKWSDAFVSDTDGQNITSKVTLTDGLEHHHHHH

SEQ ID NO: 44 - DNA, 3573 bp, Artificial Sequence
  Polynucleotide
atgggcgtggctctggaactggataagacgaaggtaaaagtaggggacataataacagcgacgataaagatagagaacatgaagaattttgcagggtaccagttgaatatcaagtatgacccgaccatgttggaggcaatagaactggagacaggaagtgcgatagcgaagaggacatggccggttacaggaggtactgttctgcaaagtgacaattatggaaagacgactgcggtagcgaatgatgtaggagcaggtataataaactttgctgaggcatactcgaaccttaccaaatacagagagacaggtgtggcagaagagacaggtataataggaaagataggcttcagagtgctgaaggcaggaagtacggctataagatttgaggatacgacagcgatgccgggagcaatagaaggaacatacatgttcgactggtatggcgagaacatcaaagggtatagcgtagtacagcctggggaaatagtggtagaaggagaagagccgggtgaagagccgacagaagagcctgtaccgacagagacatcggtagatcccacaccgacagtgacagaagagcctgtaccttcagagcttccagattcctatgctagacttaaagttacagtaggaacagctaatggtaagcctggcgatacagtaacagttcctgttacatttgctgatgtagcaaagatgaaaaacgtaggaacatgtaatttctatcttggatatgatgcaagcctgttagaggtagtatcagtagatgcaggtccaatagttaagaatgcagcagttaacttctcaagcagtgcaagcaacggaacaatcagcttcctgttcttggataacacaattacagacgaattgataactgcagacggtgtgtttgcaaatattaagttcaaattaaagagtgtaacggctaaaactacaacaccagtaacatttaaagatggtggagcttttggtgacggaactatgtcaaagatagcttcagttactaagacaaacggtagtgtaacgatcgatccgaccaagggagcaacaccaacaaatacagctacgccgacaaaatcagctacggctacgcccaccaggccatcggtaccgcggccgcatttacaggttgacattggaagtactagtggaaaagcaggtagtgttgttagtgtacctataacatttactaatgtacctaaatcaggtatctatgctctaagttttagaacaaatttcgacccacaaaaggtaactgtagcaagtatagatgctggctcactgattgaaaatgcttctgattttactacttattataataatgaaaatggttttgcatcaatgacgtttgaagccccagttgatagagctagaatcatagatagtgatggtgtatttgcaaccattaactttaaagttagtgatagtgccaaagtaggtgaactttacaatattactactaatagtgcatatacttcattctattattctggaactgatgaaatcaaaaatgttgtttacaatgatggaaaaattgaggtaattgcatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatataggatccgttcctccgaaaactaccatcattgccggttctgccgaagcgccccaaggaagtgatatccaggtgcctgttaaaatcgaaaatgctgacaaagtgggcagcataaatctcatcctgagctacccgaatgtgcttgaggttgaggatgtgcttcagggctctctaactcagaactcacttttcgattacaatgttgaaggtaatcaaattaaagttggcatcgcggacagtaacgggattagcggcgacggttcgctgttctacgtaaagttcagagttacaggcaatgaaaaagcggagcaggcagaaaacgttaaaggtaaacttaggggcttgggccaacagctctccgaaatcacactcagaaactctcacgctctcacccttcaagggatcgaaatctacgacattgatgggaattctgtaaaggttgcgacgataaatgggactttcaggattgtctctcaggaagaagctagcgccggtggtttatccgctgtgcagcctaatgttagtttaggcgaagtactggatgtttctgctaacagaaccgctgctgacggaacagttgaatggcttatcccaacagtaactgcagctccaggccagacggtcactatgcccgtagtagtcaagagttcaagtcttgcagttgctggtgcgcagttcaagatccaggcggcgacaggcgtacgttattcgtccaagacggacggtgacgcttacggttcaggcattgtgtacaataatagtaagtatgcttttggacagggtgcaggtagaggaatagttgcagctgatgattcggttgtgcttactcttgcatatacagttcccgctgattgtgctgaaggtacatatgatgtcaagtggtctgatgcgtttgtaagtgatacagacggacagaatatcacaagtaaggttactcttactgatggcctcgagcaccaccaccaccaccactga

SEQ ID NO: 45 - protein, 623 aa, Artificial Sequence
  Polypeptide
MHHHHHHTSPQVTSSPSREEPRAGTIRNPVLTGFYPDPSILRVGDDYYMATSTFEWYPGVTLHHSRDLVHWRPLGGALTETRLLDLAGRRDGAGVWAPALSYRDGLFFLVFTNVASYSGNFWDAPNYVTTAPDITGPWSDPVPLHSLGFDPSLFHDDDGRSWLLSTSMDWRPGRDAFGGIVAQEFSVRDMKLVGEPVIIFTGTEAGVTEAPHIYKRDGWYYLVTAEGGTQWEHQVTVARSRSVTGPYEVDPAGPALTSRHVPEAPLQKAGHASMVETQHGEWYFAHLTGRPMPPSGRCVLGRETALQKIEWSSDGWPRVRNAEPLLEVPGPRGLAPHPWPQPSETDHFDDPTPRPEWSTLRRPFDSSWVSLTERPGYLRIRGGQSPAGLHEPSLVARRLQHRACIFEACLEFKPEDFRQMAGITAYYNTRQWHYLRINRDDRGGVFAGVLTSDRGIIREVGRRISVTDWPKVFLRAEIDRNDLRFAVSSDGSTWADMGVRLDMSILSDEYAEERFGNDPIMWGFTGAFLGLWAHDMTGAGLPADFDFCTYRPQSPSTSPVIVYGDYNNDGNVDALDFAGLKKYIMAADHAYVKNLDVNLDNEVNAFDLAILKKYLLGMVSKLP

SEQ ID NO: 46 - DNA, 1872 bp, Artificial Sequence
  Polynucleotide
atgcaccatcaccatcaccatacttctccccaagtcacgtcctccccgtctcgtgaggaaccgagggcgggcacgattcgcaacccggtactcaccggcttctaccccgacccttccatcctgcgagtgggcgacgactactacatggcgacctccacattcgagtggtatcccggagtgaccctgcaccattcccgggacttggtgcactggcgccccctgggcggtgcactcaccgagactcgactgctggacctggctggacggcgggacggcgcaggggtgtgggcacccgccctgtcctaccgggacggactgttcttcctcgtcttcacgaacgtcgcaagctacagcggcaacttctgggacgcgcccaactacgtcaccaccgctcccgacatcaccggcccctggtccgacccggtgccgctccactccctcggcttcgacccgtcgctgttccacgacgacgacggacggagctggctgctcagcacctccatggactggcggccgggacgggacgcgttcggtggcatcgtcgcccaagagttctcggtgcgcgacatgaaactcgtcggtgaaccggtgatcatcttcaccggcaccgaagccggcgtgaccgaggcgccccacatctacaagcgcgacggctggtactacctggtcaccgccgaaggcggcacccagtgggagcaccaggtcaccgtggcccgctcccgctcggtcaccggaccctacgaggtcgacccggccgggccagccctcacctcgcggcacgttcccgaagcgccgctgcagaaggccgggcacgcgagcatggtcgaaacccagcacggcgaatggtatttcgcgcacctgaccggacgcccgatgccgcccagcggccggtgcgtcctcggtcgggagaccgcgttgcagaagatcgaatggtcttcagacgggtggccccgcgtccgcaacgcggaaccgctgctggaagtgccgggaccgcgcggcctggccccgcacccgtggccgcagccgtcggagaccgaccacttcgacgaccccacgccgcggcccgagtggagcacgctgcgccggcccttcgactcctcctgggtctccctcaccgaacggcccggctacctgcggatccgcggcgggcagtcgcctgctggcctgcacgagcccagcctggtggcacgccgactgcagcaccgcgcctgcatcttcgaagcctgcctggagttcaagccggaagacttccggcagatggcaggcatcaccgcctactacaacacccgccaatggcactacctgcggatcaaccgcgacgaccggggcggcgtgttcgcgggcgtgctcaccagcgaccgcggcatcatccgcgaagtgggacggcggatcagcgtcaccgactggccgaaggtcttcctgcgcgccgaaatcgaccggaacgacctgcgcttcgccgtctcctccgacggcagcacgtgggctgacatgggggtgcgtctggacatgagcatcctgtccgacgagtacgccgaggaacggttcggcaacgaccccatcatgtggggtttcacgggggcgttcctcggcctgtgggcccacgacatgaccggggcagggctccctgccgacttcgacttctgcacctaccggcctcagtccccctccactagtcctgtaattgtatatggagattataacaatgatggaaatgttgatgcacttgattttgcaggcttaaagaaatatattatggctgctgaccatgcttatgtaaagaatttggatgttaatctcgacaatgaagtgaatgcatttgaccttgctattttgaaaaaatatctgcttggtatggtaagtaagctaccttaa

SEQ ID NO: 47 - protein, 399 aa, Artificial Sequence
  Polypeptide
MASMHHHHHHAVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNPGGGNPPGGGNPPGGGGCTATLSAGQQWNDRYNLNVNVSGSNNWTVTVNVPWPARIIATWNIHASYPDSQTLVARPNGNGNNWGMTIMHNGNWTWPTVSCSANELTATTTPTTTPTTTPTPKFIYGDVDGNGSVRINDAVLIRDYVLGKINEFPYEYGMLAADVDGNGSIKINDAVLVRDYVLGKIFLFPVEEKEE

SEQ ID NO: 48 - DNA, 1200 bp, Artificial Sequence
  Polynucleotide
atggctagcatgcaccatcaccatcaccacgccgtgacctccaacgagaccgggtaccacgacgggtacttctactcgttctggaccgacgcgcctggaacggtctccatggagctgggccctggcggaaactacagcacctcctggcggaacaccgggaacttcgtcgccggtaagggatgggccaccggtggccgccggaccgtgacctactccgccagcttcaacccgtcgggtaacgcctacctgaccctctacgggtggacgcggaacccgctcgtggagtactacatcgtcgaaagctggggcacctaccggcccaccggtacctacatgggcacggtgaccaccgacggtggtacctacgacatctacaagaccacgcggtacaacgcgccctccatcgaaggcacccggaccttcgaccagtactggagcgtccgccagtccaagcggaccagcggtaccatcaccgcggggaaccacttcgacgcgtgggcccgccacggtatgcacctcggaacccacgactacatgatcatggcgaccgagggctaccagagcagcggatcctccaacgtgacgttgggcaccagcggcggtggaaaccccggtgggggcaacccccccggtggcggcaacccccccggtggcggtggctgcacggcgacgctgtccgcgggccagcagtggaacgaccgctacaacctcaacgtcaacgtcagcggctccaacaactggaccgtgaccgtgaacgttccgtggccggcgaggatcatcgccacctggaacatccacgccagctacccggactcccagaccttggttgcccggcctaacggcaacggcaacaactggggcatgacgatcatgcacaacggcaactggacgtggcccacggtgtcctgcagcgccaacgagctcacagcaactacaacaccaactacaacaccaactacaacaccaacgcctaaatttatatatggtgatgttgatggtaatggaagtgtaagaattaatgatgctgtcctaataagagactatgtattaggaaaaatcaatgaattcccatatgaatatggtatgcttgcagcagatgttgatggtaatggaagtataaaaattaatgatgctgttctagtaagagactacgtgttaggaaagatatttttattccctgttgaagagaaagaagaataa

SEQ ID NO: 49 - protein, 460 aa, Artificial Sequence
  Polypeptide
MASHHHHHHGPVHDHHPAPHSNAKSERLRWAAPDGFYIGSAVAGGGHHLEQDYPDPFTHDGKYRSILAQQFSSVSPENQMKWEYIHPEPDRYDFAMADKIVDFAERNDQKVRGHTLLWHSQNPEWLEEGDYSPEELREILRDHITTVVGRYAGRIHQWDVANEIFDEQGNLRTQENIWIRELGPGIIADAFRWAHEADPNAELFFNDYNVEGINPKSDAYYELIQELLDDGVPVHGFSVQGHLSTRYGFPGDLEQNLRRFDELGLATAITELDVRMDLPASGKPTPKQLEQQADYYQQALEACLAVEGCDSFTIWGFTDKYSWVPVFFPDEGAATIMTEKYERKPAFFALQQTLREARCADSPKPGPGKPKPGKGPKHDHCTSTYKVPGTPSTKLYGDVNDDGKVNSTDAVALKRYVLRSGISINTDNADLNEDGRVNSTDLGILKRYILKEIDTLPYKN

SEQ ID NO: 50 - DNA, 1383 bp, Artificial Sequence
  Polynucleotide
atggctagccatcaccatcaccatcacggaccggtccacgaccatcatcccgctccccactccaacgcgaaatccgagcggctgcgctgggctgcccccgacggcttctacatcggcagcgcggtcgcgggcggcggccaccacctggagcaggactaccccgaccccttcacccacgacgggaaataccgcagcatcctggctcagcagttcagctcagtctccccggaaaaccagatgaagtgggagtacatccatcctgagccggaccgctacgacttcgccatggccgacaagatcgtcgacttcgcggagcgtaacgaccagaaggtccgcggtcacaccctgctgtggcacagccagaaccccgagtggctcgaagagggcgactactcccctgaggagctgcgcgagatcctgcgggaccacatcaccaccgtggtcggccgctacgccggacggatccaccagtgggatgtggccaacgagatcttcgacgagcagggcaacctgcgtactcaggagaacatctggatccgcgagctcggccccggcatcatcgctgacgcgttccgctgggcgcacgaggcagacccgaacgcggagctgttcttcaacgactacaacgtggagggcatcaacccgaagagcgacgcctactacgaactcatccaggagctgctcgacgacggggttccggtccacggcttctccgtccaggggcacctgagcacccgctacggcttcccgggcgacctggaacagaacctgcgccggttcgacgagctcggtctggccacggcgatcaccgagctggacgtgcgcatggacctgccggccagcggcaagccgaccccgaagcagttggagcagcaggccgactactaccagcaggcgcttgaagcgtgcctggccgtggaaggctgcgactccttcacgatctggggcttcacggacaagtactcctgggtgccggtgttcttccccgacgagggcgcggcgacgatcatgacggagaagtacgagcgcaagcccgctttcttcgcgctgcagcagacgctgcgggaagcccggtgcgcggacagccccaagccgggaccgggcaagccgaagccgggcaagggccccaagcacgatcactgtactagtacatataaagtacctggtactccttctactaaattatacggcgacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctcaaagaaatagatacattgccgtacaagaactaa

SEQ ID NO: 51 - protein, 378 aa, Artificial Sequence
  Polypeptide
MANDSPFYVNPNMSSAEWVRNNPNDPRTPVIRDRIASVPQGTWFAHHNPGQITGQVDALMSAAQAAGKIPILVVYNAPGRDCGNHSSGGAPSHSAYRSWIDEFAAGLKNRPAYIIVEPDLISLMSSCMQHVQQEVLETMAYAGKALKAGSSQARIYFDAGHSAWHSPAQMASWLQQADISNSAHGIATNTSNYRWTADEVAYAKAVLSAIGNPSLRAVIDTSRNGNGPAGNEWCDPSGRAIGTPSTTNTGDPMIDAFLWIKLPGEADGCIAGAGQFVPQAAYEMAIAAGGTAVPEEANKGDVNGDGEINSLDALLALQMSIGKVEPNPVADMDGDGKVLAKDATEIMKMATDMMIRRTAEIISQNGLLGKLEHHHHHH

SEQ ID NO: 52 - DNA, 1137 bp, Artificial Sequence
  Polynucleotide
atggccaatgattctccgttctacgtcaaccccaacatgtcctccgccgaatgggtgcggaacaaccccaacgacccgcgtaccccggtaatccgcgaccggatcgccagcgtgccgcagggcacctggttcgcccaccacaaccccgggcagatcaccggccaggtcgacgcgctcatgagcgccgcccaggccgccggcaagatcccgatcctggtcgtgtacaacgccccgggccgcgactgcggcaaccacagcagcggcggcgcccccagtcacagcgcctaccggtcctggatcgacgaattcgctgccggactgaagaaccgtcccgcctacatcatcgtcgaaccggacctgatctcgctgatgtcgagctgcatgcagcacgtccagcaggaagtcctggagacgatggcgtacgcgggcaaggccctcaaggccgggtcctcgcaggcgcggatctacttcgacgccggccactccgcgtggcactcgcccgcacagatggcttcctggctccagcaggccgacatctccaacagcgcgcacggtatcgccaccaacacctccaactaccggtggaccgctgacgaggtcgcctacgccaaggcggtgctctcggccatcggcaacccgtccctgcgcgcggtcatcgacaccagccgcaacggcaacggccccgccggtaacgagtggtgcgaccccagcggacgcgccatcggcacgcccagcaccaccaacaccggcgacccgatgatcgacgccttcctgtggatcaagctgccgggtgaggccgacggctgcatcgccggcgccggccagttcgtcccgcaggcggcctacgagatggcgatcgccgcgggcggcaccgcggtaccagaagaagcaaacaagggagatgtgaatggagatggagaaataaacagtctcgacgctctgcttgcacttcagatgtcaatcgggaaggttgagccgaaccctgtagcagatatggatggggatggaaaggtgcttgcgaaggatgccactgaaatcatgaagatggcaacagacatgatgatcagaagaacggcggaaattataagccagaatggcttactgggtaagctcgagcaccaccaccaccaccactga

SEQ ID NO: 53 - protein, 444 aa, Artificial Sequence
  Polypeptide
MHHHHHHSTLRELAAQNGGRHFGTAIAYSPLNSDAQYRNIAATQFSAITHENEMKWESLEPQRGQYNWSQADNIINFAKANNQIVRGHTLVWHSQLPSWLNNGGFSGSQLRSIMENHIEVVAGRYRGDVYAWDVVNEAFNEDGTLRDSIWYRGMGRDYIAHAFRKAHEVDPDAKLYINDYNIEGINAKSNGLYNLVVDLLRDGVPIHGIGIQSHLIVGQVPSTFQQNIQRFADLGLDVAITELDIRMQMPADQYKLQQQARDYEAVVNACLAVTRCIGITVWGIDDERSWVPYTFPGEGAPLLYDGQYNRKPAWYAVYEALGGDSSGGGPGEPGGPGGPGEPGGPGGPGEPGGPGDGTSGTKLVPTWGDTNCDGVVNVADVVVLNRFLNDPTYSNITDQGKVNADVVDPQDKSGAAVDPAGVKLTVADSEAILKAIVELITLPQ

SEQ ID NO: 54 - DNA, 1335 bp, Artificial Sequence
  Polynucleotide
atgcaccatcaccatcaccactcgaccctgcgggaactggctgcccagaacggcggccgccacttcggtacggctatcgcctacagcccgctcaacagtgacgcccagtaccgcaacatcgcggctacccagttcagcgccatcacccacgaaaacgagatgaagtgggagtcgctggagccgcagcggggccagtacaactggagccaggccgacaacatcatcaacttcgccaaggccaacaaccagattgtgcgcggccacaccctggtctggcacagccagctgccgtcctggctgaacaacggcggcttctccggcagccagctccggtccatcatggagaaccacatcgaggtggtggccggacgctaccggggtgacgtctacgcctgggacgtggtcaacgaagcgttcaacgaggacggtacgctccgcgactcgatctggtaccgcggcatgggtcgcgactacatcgcccacgcgttccgcaaggcgcacgaggtcgaccccgacgccaagctgtacatcaacgactacaacatcgaaggcatcaacgctaagagcaacggcctctacaacctggtggtcgacctgctccgcgacggtgtgccgatccacggtatcggtatccagtcccacctgatcgtcggccaggtgccgtccacgttccagcagaacatccagcggttcgctgacctcggcctggacgtggccatcaccgagctggacatccgcatgcagatgccggccgaccagtacaagctccagcagcaggcccgcgactacgaggccgtggtcaacgcctgcctcgcggtgacccgctgcatcggtatcaccgtctggggtatcgacgacgagcgctcctgggtgccctacaccttcccgggtgaaggtgctccgctgctctacgacggccagtacaaccgcaagcccgcctggtacgcggtctacgaggctctcggcggcgactcctccggcggcggtccgggtgagccgggcggtcctggcggtccgggtgagccgggcggtcctggcggtccgggtgaaccgggcggccccggtgacggcactagtggcacaaagctcgttcctacatggggcgatacaaactgcgacggcgttgtaaatgttgctgacgtagtagttcttaacagattcctcaacgatcctacatattctaacattactgatcagggtaaggttaacgcagacgttgttgatcctcaggataagtccggcgcagcagttgatcctgcaggcgtaaagctcacagtagctgactctgaggcaatcctcaaggctatcgttgaactcatcacacttcctcagtaa